(19) United States
(12) Patent Application Publication
(10) Pub. No.: US 2023/0364031 A1
(43) Pub. Date: Nov. 16, 2023

(54) TREATMENT OF BRAIN COPPER DISORDERS

(71) Applicants: Garth COOPER, Auckland (NZ); Sasha A. PHILBERT, London (GB)

(72) Inventors: Garth COOPER, Auckland (NZ); Sasha A. PHILBERT, London (GB)

(21) Appl. No.: 18/136,797

(22) Filed: Apr. 19, 2023

Related U.S. Application Data
(60) Provisional application No. 63/332,551, filed on Apr. 19, 2022.

(51) Int. Cl. A61K 31/132; A61K 45/06; A61P 25/28

(52) U.S. Cl. CPC A61K 31/132; A61K 45/06; A61P 25/28

(57) ABSTRACT

The present disclosure relates to the treatment and prevention of impaired cognitive function, cognitive decline and/or dementia or signs of cerebral neurodegeneration in a subject with diabetes mellitus by administration of a pharmaceutical composition comprising a compound capable of lowering the level or amount of copper in the hippocampus, or elsewhere in the brain, of the subject.

This application claims priority to U.S. Provisional Patent Application No. 63/332,551 filed Apr. 19, 2022, the entire contents of which are incorporated herein by reference for all purposes.

### FIELD

The invention concerns diabetes, dementia, impaired cognitive function, copper binding agents and compounds capable of normalizing copper metabolism.

### INCORPORATION BY REFERENCE

All U.S. patents, U.S. patent application publications, U.S. patent applications (including provisional patent applications), foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content is repeated in the application and will be treated as part of the text and content of this application as filed.

### BACKGROUND

The following includes information that may be useful in understanding the present inventions. It is not an admission that any of the information is prior art, or relevant, to the presently described or claimed inventions, or that any publication or document that is specifically or implicitly referenced is prior art or a reference that may be used in evaluating patentability of the described or claimed inventions.

Essential metals are vital for the normal physiological functioning of biological systems. They are often present in metalloprotein/metalloenzyme complexes and mediate a variety of processes, including mitochondrial function, transcriptional regulation, and cell metabolism. However, the presence of essential metals at concentrations outside their physiological ranges can lead to severe cellular dysfunction.

Copper is an essential trace element involved in a large number of biological processes in living cells. Analysis of the human proteome has identified 54 copper-binding proteins, of which 12 are copper transporters, approximately half are enzymes and one (Antioxidant 1 Copper Chaperone, ATOX1) is a transcription factor. The majority of copper in the body is located in organs with high metabolic activity, such as liver, kidneys, heart and brain, with approximately 5% of total copper in the serum, of which up to 95% is bound to ceruloplasmin.

Copper is also involved in oxidative stress, and unbound copper behaves as a potent oxidant, catalyzing the formation of highly reactive hydroxyl radicals leading to DNA, protein and lipid damage. Therefore, cellular copper concentration needs to be finely regulated by complex homeostatic mechanisms of absorption, excretion and bioavailability.

Wilson's disease and Menkes' disease are genetically-transmitted disorders of copper metabolism caused respectively by defects in the genes encoding the ATP-linked cell copper transporters ATP7B and ATP7A (de Bie P, et al. Molecular pathogenesis of Wilson and Menkes disease: correlation of mutations with molecular defects and disease phenotypes. J Med Genet 2007; 44: 673-88). Excess brain copper, such as occurs in Wilson's Disease (Cumings J N. The copper and iron content of brain and liver in the normal and in hepato-lenticular degeneration. Brain 1948; 71(Pt. 4): 410-5) and deficient brain copper, as in Menkes' disease (Walker-Smith J A, et al. Therapeutic implications of copper deficiency in Menkes's steely-hair syndrome. Arch Dis Child 1973; 48: 958-62) are both known to cause severe neurodegeneration if untreated by pharmacological restoration of brain copper levels. Wilson's disease and Menkes' disease serve as models for the impact of patterns of defective copper homeostasis on the brain as in each disease the disease mechanism is known to be defective copper homeostasis leading to tissue damage, caused by copper overload and copper deficiency, respectively.

Regarding copper and diabetes mellitus, some have reported a copper increase in the urine or serum of people with type 2 diabetes. Cooper, G. J., et al. (2005) Diabetes 54, 1468-147. On the other hand, clinical studies comparing plasma or serum copper levels in patients with diabetes and in healthy individuals report conflicting findings (Qiu, Q., et al. Copper in Diabetes Mellitus: a Meta-Analysis and Systematic Review of Plasma and Serum Studies. Biol Trace Elem Res 177, 53-63 (2017)). See also Lowe, J., et al. Dissecting Copper Homeostasis in Diabetes Mellitus. IUBMB Life 69(4): 255-262 (2017).

Likewise, although defective brain-metal metabolism occurs in various age-related neurodegenerative diseases, there is no consensus concerning the role of such impaired metal homeostasis in their pathogenesis Alzheimer's disease is a neurodegenerative disorder that is characterized by amyloid plaques in patient brain tissue. The plaques are mainly made of β-amyloid peptides and trace elements including Zn²⁺, Cu²⁺, and Fe²⁺. Some have proposed that Alzheimer's disease can be considered a type of metal dyshomeostasis, and numerous studies have focused on copper ions, which seem to be one of the main cationic elements in plaque formation. However, the involvement of copper in Alzheimer's disease is not settled, with some studies showing a copper deficiency, while other data point to copper overload. Bogheri S., et al., Role of Copper in the Onset of Alzheimer's Disease Compared to Other Metals, Front Aging Neurosci. 2017; 9: 446. See Drew, S. C., (2017) The Case for Abandoning Therapeutic Chelation of Copper Ions in Alzheimer's Disease, Front. Neurosci. 11:317.

Similarly, while many studies associate an increase in the levels of free copper with increased Parkinson's disease, another neurodegenerative disorder, this correlation remains unproven. Various studies support the hypothesis that both copper increase (leading to enhancement of intracellular oxidative conditions) and copper deficiency (leading to formation of superoxide dismutase 1 aggregates) are correlated to an enhanced risk of developing the disease. Marco Bisaglia and Luigi Bubacco, Copper Ions and Parkinson's Disease: Why Is Homeostatsis So Relevant? Biomolecules. 2020 February; 10(2): 195. See Ajsuvakova O. P., et al. Assessment of copper, iron, zinc and manganese status and speciation in patients with Parkinson's disease: A pilot study. J. Trace Elem. Med. Biol. 2019:126423.

According to a 2016 study, people who have type 2 diabetes are up to about 60 percent more likely to develop Alzheimer's disease or another type of dementia, such as vascular dementia, compared with those without diabetes. Chatterjee S, et al., Type 2 Diabetes as a Risk Factor for Dementia in Women Compared With Men: A Pooled Analysis of 2.3 Million People Comprising More Than 100,000 Cases of Dementia, Diabetes Care 2016 February; 39(2): 300-307. Some have even proposed that Alzheimer's disease should also be classified as a type of diabetes. The term “type 3 diabetes” has been proposed to describe the hypothesis that Alzheimer's disease, which is a major cause of dementia, is triggered by a type of insulin resistance and insulin-like growth factor dysfunction that occurs specifically in the brain. This condition also has been used by some to describe people who have type 2 diabetes and are also diagnosed with Alzheimer's disease dementia.

According to scientists at the Mayo Clinic, there's already an established link between Alzheimer's and type 2 diabetes. “Researchers link Alzheimer's gene to Type 3 diabetes” (Oct. 25, 2017) www.newsnetwork.mayoclinic.org/discussion/researchers-link-alzheimers-gene-to-type-iii-diabetes, and it's been suggested that Alzheimer's may be triggered by insulin resistance in the brain, with some saying that Alzheimer's is simply “diabetes in your brain.” See de la Monte S M and Wands J R, Alzheimer's Disease Is Type 3 Diabetes—Evidence Reviewed, J Diabetes Sci Technol. 2008 November; 2(6): 1101-1113 (concluding that the term “type 3 diabetes” accurately reflects the fact that Alzheimer's disease represents a form of diabetes that selectively involves the brain and has molecular and biochemical features that overlap with both type 1 and type 2 diabetes mellitus).

There is robust evidence for increased rates of lacunar infarction and cerebral atrophy in type 2 diabetes (de Bresser J, et al. Progression of cerebral atrophy and white matter hyperintensities in patients with type 2 diabetes. Diabetes Care 2010; 33(6): 1309-14; van Harten B, et al. Brain imaging in patients with diabetes: a systematic review. Diabetes Care 2006; 29(11): 2539-48) where the hippocampus is often severely affected (Gold S M, et al. Hippocampal damage and memory impairments as possible early brain complications of type 2 diabetes. Diabetologia 2007; 50(4): 711-9; Li M, et al. Atrophy patterns of hippocampal subfields in T2DM patients with cognitive impairment. Endocrine 2020; 68: 536-48), even at early stages (Dong S, et al. Individuals in the prediabetes stage exhibit reduced hippocampal tail volume and executive dysfunction. Brain Behav 2019; 9(8): e01351). Gold et al. found deficits in hippocampal-based memory performance and preservation of other cognitive domains. Relative to control subjects, they reported, individuals with diabetes had reductions in brain volumes that were restricted to the hippocampus, with an inverse relationship between glycemic control and hippocampal volume.

In addition to hippocampal atrophy in type 2 diabetes, hippocampal atrophy is also a prominent early aspect of neurodegeneration in sporadic Alzheimer's disease (Dubois B, Feldman H H, Jacova C, et al. Advancing research diagnostic criteria for Alzheimer's disease: the IWG-2 criteria. Lancet Neural 2014; 13: 614-29), the most common form of dementia, where hippocampal-Cu levels are severely lowered to values similar to reported brain-Cu levels in Menkes' disease (Walker-Smith J A, et al. Therapeutic implications of copper deficiency in Menkes's steely-hair syndrome. Arch Dis Child 1973; 48: 958-62). However, copper levels in cerebrospinal fluid of AD patients are 2.2-fold higher than in controls, and increased levels of ceruloplasmin in the brain and in cerebrospinal fluid have been observed. Basun H., et al. Metals and trace elements in plasma and cerebrospinal fluid in normal aging and Alzheimer's disease. J. Neural. Transm. Park Dis. Dement. Sect. 1991; 3:231-258.

Longitudinal epidemiological studies have implicated chronic hyperglycemia and microvascular disease in the pathogenesis of diabetes-related cognitive dysfunction, but the causal pathway that underlies the statistical associations between type 2 diabetes and dementia is unknown (McCrimmon R J, et al., Diabetes and cognitive dysfunction. Lancet 2012; 379: 2291-9).

We have discovered, unexpectedly, that brain hippocampal copper is markedly elevated in type 2 diabetes, approximating literature values in Wilson's disease (a neurodegenerative disease of copper excess), whereas, contrastingly, we found that hippocampal copper values in the brains of sporadic Alzheimer's disease patients are severely deficient, as in Menkes disease (a neurodegenerative disease of copper deficit). We also discovered that elevation in hippocampal copper levels was the only substantive perturbation in essential-metal homeostasis in the brain of patients with type 2 diabetes with no differences in tissues from the frontal or temporal cortices, or meninges.

Therapeutic approaches to lower hippocampal copper levels are disclosed and claimed for treating patients with diabetes or vascular dementia who show or are at risk for impaired cognitive function, cognitive decline, or show signs of cerebral neurodegeneration and/or dementia.

### BRIEF SUMMARY

The inventions described and claimed herein have many attributes and embodiments including, but not limited to, those set forth or described or referenced in this Brief Summary. It is not intended to be all-inclusive, and the inventions described and claimed herein are not limited to or by the features or embodiments identified in this introduction, which is included for purposes of illustration only and not restriction.

The invention relates to methods of treating or preventing impaired cognitive function, cognitive decline, or symptoms of cerebral neurodegeneration or dementia in a subject with diabetes mellitus, comprising administering to the subject a pharmaceutical composition comprising a compound capable of reducing, lowering or normalizing copper levels and/or copper metabolism. In some embodiments of the methods, the compound is a copper antagonist, such as a copper-depriving agent, a copper sequestering agent or copper removing agent. In some embodiments, the compound is a copper chelator. In some embodiments, the methods of the invention bring the copper values in a subject to within normal ranges. In some embodiments, the methods of the invention bring the copper values in a subject to within 75-110% of normal ranges. In some embodiments, the methods of the invention bring hippocampal copper to within normal expected ranges, or at least to lower copper or to reduce copper to within a range that reduces impaired cognitive function, cognitive decline, or symptoms of cerebral neurodegeneration or dementia. In some embodiments, the methods of the invention are used to bring copper levels in the blood of a subject to within about 70 to 140 micrograms per deciliter (mcg/dL). Copper levels may be evaluated and monitored using urinalysis, for example. Serum and fecal copper measurements are also available, although urine copper measurements are preferred.

In some embodiments, the methods of treating or preventing impaired cognitive function, cognitive decline, or symptoms of cerebral neurodegeneration or dementia in a subject with diabetes mellitus comprise administering a composition comprising or consisting essentially of a copper chelator or other copper binding compound or copper removing agent to the subject. In some embodiments, the composition lowers total copper in the subject. In some embodiments, the composition lowers copper values in the subject. In some embodiments, the composition lowers the amount of copper(II) and/or copper(I) in the subject. In some embodiments, the composition lowers excess copper(II) and/or copper(I) in the subject. In some embodiments, the composition lowers excess hippocampal copper in the subject. In some embodiments, the excess hippocampal copper in the subject is copper(II). In some embodiments, the excess hippocampal copper in the subject is copper(I). In some embodiments, the composition chelates copper(II) and copper(I) in the subject. In some embodiments of the methods, the active ingredient in the composition is a trientine. In some embodiments, the trientine is triethylenetetramine disuccinate. In some embodiments, the trientine is triethylenetetramine dihydrochloride or triethylenetetramine tetrahydrochloride.

In some embodiments of the methods, the subject of treatment in the preceding methods has vascular dementia, with or without diabetes, e.g., with or without type 1 diabetes, with or without type 2 diabetes, with or without type 3 diabetes and/or with or without type 4 diabetes.

In some embodiments of the methods, one or more symptoms of the treated condition, e.g., impaired cognitive function, cognitive decline, or symptoms of cerebral neurodegeneration and/or dementia is/are reduced or alleviated. In some method embodiments, one or more symptoms of the treated condition, e.g., impaired cognitive function and/or dementia, etc., is/are substantially eliminated.

In some embodiments, methods of the invention are used for the treatment of fronto-temporal dementia (FTD). In some embodiments of the methods, one or more symptoms of fronto-temporal dementia is/are reduced or alleviated. In some method embodiments, one or more symptoms of fronto-temporal dementia is/are substantially eliminated.

In some embodiments, methods of the invention are used for the treatment of fronto-temporal lobar dementia (FTLD). In some embodiments of the methods, one or more symptoms of fronto-temporal lobar dementia is/are reduced or alleviated. In some method embodiments, one or more symptoms of fronto-temporal lobar dementia is/are substantially eliminated.

In some embodiments, methods of the invention are used for the treatment of amyotrophic lateral sclerosis (ALS) and/or accompanying motor neuron disease (MND) dementia. In some embodiments of the methods, one or more symptoms of ALS and/or MND is/are reduced or alleviated. In some method embodiments, one or more symptoms of ALS and/or MND is/are substantially eliminated.

In some embodiments, methods of the invention are used for the treatment of dementia with Lewy bodies (DLB). In some embodiments of the methods, one or more symptoms of DLB is/are reduced or alleviated. In some method embodiments, one or more symptoms of DLB is/are substantially eliminated.

In some embodiments, methods of the invention are used for the treatment of multiple sclerosis (MS). In some embodiments of the methods, one or more symptoms of MS is/are reduced or alleviated. In some method embodiments, one or more symptoms of MS is/are substantially eliminated.

In some embodiments, methods of treating FTD, FTLD, ALS, MND, DLB and MS comprises administering to the subject a pharmaceutical composition comprising a compound capable of normalizing copper metabolism. In some embodiments, the compound capable of reducing or lowering copper values, copper levels or total copper or capable of normalizing copper values, copper levels, total copper or copper metabolism is a copper chelator or other copper binding or copper removing agent. Other embodiments of these methods, and agents, compounds, compositions, and procedures useful in these methods, are described herein.

In some embodiments of the methods, the subject has diabetes and dementia and/or cognitive decline and/or cognitive and/or memory impairment. In some embodiments, the subject has type 2 diabetes. In other embodiments, the subject has type 1 diabetes. In some embodiments, the subject has type 3 diabetes. In some embodiments, the subject has type 4 diabetes.

In some embodiments, the subject being treated has type 1 diabetes and one or more of the cognitive domains negatively affected by cognitive impairment in the subject is a reduction in overall cognition, fluid and crystallized intelligence, speed of information processing, psychomotor efficiency, visual and sustained attention, mental flexibility, and/or visual perception.

In some embodiments, the subject being treated has type 2 diabetes and one or more of the cognitive domains negatively affected by cognitive impairment in the subject is memory (verbal memory, visual retention, working memory, immediate recall, delayed recall), psychomotor speed and frontal lobe/executive function, processing speed, complex motor function, verbal fluency and/or attention.

In some embodiments, the cognitive impairment prevents an individual with diabetes, for example, type 2 diabetes, from concentrating, recalling memories, and/or leads to mental fatigue.

In some embodiments of the methods of the invention, structural correlates of diabetes-related cognitive impairment are assessed with brain magnetic resonance imaging (MRI). MRI may be used to assess a subject prior to and/or during treatment. In some embodiments, in evaluating cognitive impairment in patients with diabetes for treatment as described herein, e.g., in patients with type 2 diabetes, MRI is used to examine or confirm the presence of normal or abnormal cerebral structure before and/or during treatment.

In some embodiments, in patients with type 2 diabetes, for example, white matter hyperintensities are correlated with reduced performance on tests of attention, executive function, information processing speed, and memory and provide a structural basis for treatment with compounds of the invention. In some embodiments, MRI is used to examine or confirm the presence of white matter hyperintensities before and/or during treatment.

In some embodiments, MRI is used to demonstrate or confirm that subjects with diabetes, e.g., type 2 diabetes, have lacunar infarction(s), hippocampal atrophy and/or amygdala atrophy or other hippocampal defects, including hippocampal atrophy patterns, prior to treatment with compounds of the invention. In some embodiments, MRI is used to evaluate subjects with diabetes, e.g., type 2 diabetes, for hippocampal and/or amygdala atrophy or other hippocampal defects, including hippocampal atrophy patterns, during treatment with compounds of the invention.

In some embodiments, MRI is used to assess hippocampal and/or amygdala atrophy in subjects with type 2 diabetes prior to, during and/or after treatment according to methods of the invention.

In some embodiments, the invention relates to methods of treating or preventing impaired cognitive function in a subject with vascular dementia, also sometimes referred to as vascular cognitive impairment, or VCI, comprising administering to the subject a pharmaceutical composition comprising a compound capable of normalizing copper metabolism. In some embodiments, the subject may or may not have diabetes mellitus, e.g., may or may not have type 1 diabetes, may or may not have type 2 diabetes, may or may not have type 3 diabetes and may or may not have type 4 diabetes. In some embodiments, the compound capable of reducing or lowering copper values, copper levels or total copper or capable of normalizing copper values, copper levels, total copper or copper metabolism is a copper chelator or other copper binding or copper removing agent. In some embodiments, the subject with vascular dementia or VCI has problems with reasoning, planning, judgment, memory and other thought processes. In some embodiments, the composition lowers total copper in the subject. In some embodiments, the composition lowers copper values in the subject. In some embodiments, the composition lowers the amount of copper(II) and/or copper(I) in the subject. In some embodiments, the composition lowers excess copper(II) and/or copper(I) in the subject. In some embodiments, the composition lowers excess hippocampal copper in the subject. In some embodiments, the excess hippocampal copper in the subject is copper(II). In some embodiments, the excess hippocampal copper in the subject is copper(I). In some embodiments, the composition chelates copper(II) and/or copper(I) in the subject. In some embodiments, the active ingredient in the composition is a trientine. In some embodiments, the trientine is triethylenetetramine disuccinate, triethylenetetramine dihydrochloride and/or triethylenetetramine tetrahydrochloride.

In some embodiments of the methods of the invention, the compound capable of normalizing copper metabolism is capable of reducing or lowering elevated copper in a subject.

In some embodiments of the methods of the invention, the copper antagonist, e.g., a copper-lowering/removing or copper-normalizing compound in the composition binds copper²⁺. In some embodiments, the copper antagonist compound in the composition chelates copper²⁺. In some embodiments of the methods of the invention, the copper removing agent in the composition administered to the subject binds copper²⁺. Total copper, copper values and/or hippocampal copper in the subject are thereby lowered.

In some embodiments of the methods of the invention, the compound administered that is effective to lower total copper or the copper values content in a subject is a copper chelating compound. In some embodiments, the compound administered that is effective to lower total copper or the copper values content in the subject comprises or consists essentially of or consists of an agent that binds or chelates copper(I). In other embodiments, the compound administered that is effective to lower total copper or the copper values content in the subject comprises or consists essentially of or consists of an agent that binds or chelates copper(II). In other embodiments, the agent administered that is effective to lower total copper or the copper values content in the subject comprises or consists essentially of or consists of an agent that binds or chelates both copper(I) and copper(II).

In one embodiment, the compound effective to reduce copper, lower total copper or lower the copper values content in a subject and thus hippocampal copper comprises or consists essentially of or consists of a compound selected from the group consisting of D-penicillamine; N-acetylpenicillamine; triethylenetetramine (also called TETA, TECZA, trien, triene and trientine), and pharmaceutically acceptable salts thereof; trithiomolybdate, tetrathiomolybdate, ammonium tetrathiomolybdate, choline tetrathiomolybdate; bis-choline tetrathiomolybdate (thiomolybdate USAN, trade name Decuprate), 2,2,2 tetramine tetrahydrochloride; 2,3,2 tetramine tetrahydrochloride; ethylenediaminetetraacetic acid salts (EDTA, a non-preferred non-specific metal binder, administered with care to avoid toxicity); diethylenetriaminetetraacetic acid (DPTA, a non-preferred non-specific metal binder, administered with care to avoid toxicity that is due to chelation of essential metals, such as Zn and Mn); 5,7,7′12,14,14′hexaxmethyl-1,4,8,11 tetraazacyclotretradecane; 1,4,8,11 tetraazacyclotretradecane, including cyclam S, cylams, and copper-chelating cyclam derivatives, e.g., Bn-cyclam-EtOH, oxo-cyclam-EtOH and oxo-Bn-cyclam-EtOH, (HOCH₂CH₂CH₂)₂(PhCH₂)₂Cyclam and (HOCH₂CH₂CH₂)₂(4-CF₃PhCH₂)₂Cyclam; 1,4,8,11-tetraazacyclotetradecane-1,4,8,11-tetraacetic acid; 1,4,8,11-tetraazabicyclo[6.6.2]hexadecane; 4,11-bis(N,N-diethyl-amidomethyl)-1,4,8,11-tetraazabicyclo[6.6.2]hexadecane; 4,11-bis(amidoethyl)-1,4,8,11-tetraazabicyclo[6.6.2]hexadecane; melatonin; cyclic 3-hydroxymelatonin (3 OHM); N(1)-acetyl-N(2)-formyl-5-methoxykynuramine (AFMK); N(1)-acetyl-5-methoxykynuramine (AMK); N,N′-diethyldithiocarbamate; bathocuproinedisulfonic acid; bathocuprinedisulfonate; trimetazidine; triethylene tetramine tetrahydrochloride; 2,3,2-tetraamine; 1,10-orthophenanthroline; 3,4-dihydroxybenzoic acid; 2,2′-bicinchinonic acid; diamsar; 3,4′,5, trihydroxystilbene (resveratrol); mercaptodextran; disulfiram (Antabuse); sarcophagine; DiAmSar; diethylene triamine pentaacetic acid; and calcium trisodium diethylenetriaminepentaacetate; neocuproine; bathocuproine; and carnosine. In some embodiments, the compound or agent to reduce copper amounts and/or levels, lower total copper or lower the copper values content preferentially binds Cu¹⁺. In another embodiment, the compound or agent to reduce copper amounts and/or levels, lower total copper or lower the copper values content preferentially binds Cu²⁺. In some embodiments, the compound or agent to reduce copper amounts and/or levels, lower total copper or lower the copper values content that preferentially binds Cu²⁺ is triethylenetetramine disuccinate. In another embodiment, the compound or agent binds both Cu¹⁺ and Cu²⁺. In some embodiments, the compound or agent to reduce copper amounts and/or levels, lower total copper or lower the copper values content preferentially binds both Cu¹⁺ and Cu²⁺ is a penicillamine copper chelator. In some embodiments, the penicillamine copper chelator is D-penicillamine.

In some embodiments, the agent is administered to reduce total and/or hippocampal copper in the subject. In some embodiments, the pharmaceutical composition used in methods of the invention comprises a therapeutically effective amount of a triethylenetetramine and a pharmaceutically acceptable carrier, glidant, diluent, or excipient. In some embodiments, the triethylenetetramine is in the form of a pharmaceutically acceptable salt.

In another embodiment, administration of the compound or agent to reduce total and/or hippocampal copper maintains total copper in the subject within the normal human serum or plasma range of about 0.8-1.2 milligrams/L, or about 10-25 micromoles/L. In another embodiment, the agent to reduce copper maintains total copper in the subject within at least about 70% of the normal range of about 0.8-1.2 milligrams/L or about 10-25 micromoles/L, e.g., at least about 75%. In another embodiment, the compound or agent to reduce copper maintains total copper in the subject within about 75% to about 85%, or about 85% to about 95% the normal range of copper in human plasma or serum. In one aspect of the methods of the invention, the copper status of a subject provided a compound or agent to reduce copper is determined by evaluating copper in the urine of the subject. In some embodiments, the copper status of a subject provided a compound or agent to reduce copper is determined by evaluating copper in the plasma of the subject. In some embodiments, the copper status of a subject provided a compound or agent to reduce copper is determined by evaluating copper in the liver of the subject.

In one embodiment, the triethylenetetramine is a hydrochloride salt of triethylenetetramine. In some embodiments, the triethylenetetramine is a succinate salt of triethylenetetramine.

In one aspect of the invention, the method employs a pharmaceutical composition comprising substantially pure triethylenetetramine disuccinate and a pharmaceutically acceptable excipient. In another aspect, the method employs a pharmaceutical composition comprising substantially pure triethylenetetramine dihydrochloride and/or tetrahydrochloride and a pharmaceutically acceptable excipient.

In one aspect of the invention, the method employs a pharmaceutical composition comprising a crystalline form of triethylenetetramine disuccinate or crystalline form of a hydrochloride salt of triethylenetetramine. In another aspect of the invention, the method employs a pharmaceutical composition comprising triethylenetetramine disuccinate anhydrate or a hydrochloride salt of triethylenetetramine anhydrate.

In certain embodiments, the triethylenetetramine succinate salt is a triethylenetetramine disuccinate polymorph. In certain embodiments, the triethylenetetramine hydrochloride salt is a triethylenetetramine hydrochloride polymorph.

In some embodiments of the methods of the invention, the method comprises administering to the subject a therapeutically effective amount of compound selected from the group consisting of a trientine, a succinic acid addition salt of triethylenetetramine, a hydrochloric acid addition salt of triethylenetetramine, and pharmaceutically acceptable salts of D-penicillamine, N-acetylpenicillamine, tetrathiomolybdate, ammonium tetrathiomolybdate, and choline tetrathiomolybdate. The compounds reduce total copper, copper values and/or hippocampal copper in the treated subject, and one or more symptoms of impaired cognitive function, cognitive decline and/or dementia, etc., as described herein, including vascular dementia. Reductions in total copper or copper values in a subject lead to and are surrogates for reductions in hippocampal copper.

In some embodiments of the methods of the invention, the subject shows signs of cerebral degeneration or impaired cognitive function prior to treatment. In other embodiments, the subject has or is at risk for cerebral degeneration or impaired cognitive function or having cognitive decline and/or dementia. Risk for these conditions can be determined by measuring copper levels in the subject. In some embodiments, copper is measured in the urine of a subject prior to treatment during treatment or both. Other risk factors are diagnosed diabetes with shrinkage of the hippocampus and/or amygdala as shown by MRI or decreased glucose uptake in the hippocampus and/or amygdala as shown by positron emission tomography (PET) scanning.

In some embodiments, the subject with diabetes or vascular dementia has elevated urinary copper output. In some embodiments, the subject with diabetes or vascular dementia has diminished spatial memory or ability to remember directions, locations, and orientations. In some embodiments, the subject with diabetes or vascular dementia has elevated urinary copper output and diminished spatial memory or ability to remember directions, locations, and orientations.

In some embodiments of methods of the invention, the method further comprises administering an additional therapeutic agent or agents selected from an anti-inflammatory agent, an agent for treating cardiovascular disease, an agent for treating hypertension, an agent for treating kidney disease, an agent for treating depression, and an agent for treating type 2 diabetes and/or dementia.

In some embodiments, the additional therapeutic agent or agents for treating type 2 diabetes is/are selected from the group consisting of alpha-glucosidase inhibitors, biguanides, dopamine agonists, dipeptidyl peptidase-4 (DPP-4) inhibitors, glucagon-like peptide-1 receptor agonists, meglitinides, sodium-glucose transporter (SGLT) 2 inhibitors, sulfonylureas and thiazolidinediones.

In some embodiments, the additional therapeutic agent or agents for treating dementia is/are selected from the group consisting of cholinesterase inhibitors, antibodies that target the amyloid 13 protein (a biomarker of Alzheimer disease and other dementias) and N-methyl-D-aspartate (NMDA) receptor antagonists. In some embodiments, the cholinesterase inhibitor is donepezil (Aricept), galantamine (Razadyne, Razadyne ER, Reminyl) or Rivastigmine (Exelon). In some embodiments, the antibody that targets amyloid 13 protein is Aducanumab-avwa (Aduhelm). In some embodiments, the NMDA receptor antagonist is memantine (Axura, Ebixa, Namenda, etc.).

In some embodiments, the subject is a human.

In some embodiments, the pharmaceutical composition is administered orally in the form of a capsule or tablet.

In some embodiments, the compound is triethylenetetramine dihydrochloride and is administered in an amount of about 1200 mg daily. In some embodiments, the 1200 mg of triethylenetetramine dihydrochloride is administered BID in 600 mg divided doses, TID in 400 mg divided doses, or QID in 300 mg divided doses.

In some embodiments, the compound is triethylenetetramine disuccinate and is administered in a dose ranging from about 2400 mg per day to about 3000 mg per day, or more. In some embodiments, the compound is triethylenetetramine disuccinate and is administered in an amount of about 2800 mg per day. Other useful doses of triethylenetetramine disuccinate are given to equal about 1050 mg/day to about 2300 mg/day, about 1400 mg/day to about 3500 mg/day, about 2400 mg/day to about 3200 mg/day, and about 2800 mg/day to about 5600 mg/day. In some embodiments, these daily triethylenetetramine disuccinate amounts are administered in divided doses.

The invention also provides a kit for the therapeutic treatment of treating or preventing impaired cognitive function in a subject with diabetes, comprising: a) a pharmaceutical composition comprising a copper antagonist compound capable of reducing or lowering copper in the subject, e.g., reducing or lowering copper values, reducing or lowering total copper, reducing or lowering copper levels and/or amounts (e.g., urinary copper levels and/or amounts) and/or normalizing copper metabolism, including one or more of the compounds described herein, including copper(I) and/or copper(II) chelators; and b) instructions for use in the therapeutic treatment of or prevention of impaired cognitive function, cognitive decline and/or dementia, etc., as described herein, in a subject with diabetes. In some embodiments, the subject has type 1, type 2, type 3 or type 4 diabetes mellitus. In some embodiments, the subject of the instructions has vascular dementia, with or without diabetes.

In some embodiments, the compound in the kit is selected from the group consisting of triethylenetetramine dihydrochloride, triethylenetetramine tetrahydrochloride and triethylenetetramine disuccinate. In some embodiments, the triethylenetetramine disuccinate in the kit is triethylenetetramine disuccinate anhydrate.

In one aspect, the invention comprises an article of manufacture comprising a package insert instructing the user to administer the copper antagonist compound capable of reducing or lowering copper in the subject, e.g., reducing or lowering copper values, reducing or lowering total copper and/or normalizing copper metabolism, including one or more of the compounds described herein, including copper(I) and/or copper(II) chelators, to a patient with diabetes and a condition or disorder characterized by dementia (or risk for dementia). The condition or disorder characterized by dementia in the patient with diabetes may be Alzheimer's disease. The condition or disorder characterized by dementia in the patient with diabetes may be vascular dementia.

In a further aspect, the co-existing disease in a dementia patient (or a patient at risk for dementia) treatable with a compound capable of normalizing copper metabolism (e.g., one or more of the copper chelator described herein) is characterized by excess copper, such as type 2 diabetes. In a further aspect, the co-existing disease in a dementia patient treatable with a compound capable of normalizing copper metabolism (e.g., one or more of the copper chelators described herein) is characterized by a copper deficiency, such as Alzheimer's disease. In another aspect, the disease, condition or disorder is selected from the group consisting of diabetes mellitus, Alzheimer's disease, and Parkinson's disease.

A preferred pharmaceutical composition for use in the methods of the invention comprises or consists essentially of or consists of substantially pure triethylenetetramine disuccinate. Another preferred composition comprises or consists essentially of or consists of substantially pure triethylenetetramine disuccinate anhydrate. Another preferred composition is a composition that comprises or consists essentially of or consists of substantially pure triethylenetetramine disuccinate crystal having alternating layers of triethylenetetramine molecules and succinate molecules.

In another aspect of the invention, the methods of the invention maintain copper levels with about 70% to about 110% of normal in the subject, thereby eliciting by a lowering of copper values in a mammalian patient and/or reducing or lowering the level of hippocampal copper.

Both the foregoing summary and the following detailed description are exemplary and explanatory. They are intended to provide further details of the invention but are not to be construed as limiting. Other objects, advantages, and novel features will be apparent to those skilled in the art from the following detailed description of the invention.

### BRIEF DESCRIPTION OF DRAWINGS

FIG. 1 shows the concentrations of nine essential elements (A-I) in four human-brain regions compared between control (red) and T2D (green) post-mortem tissue. Data are means±95% Cl. In MN data, a single outlier from both Mg and Cu analyses was removed from the plots for clarity: this did not change the results or conclusions. Non-standard abbreviations: FC, frontal cortex; TC, temporal cortex; HP, hippocampus; MN, meninges.

FIG. 2 shows data from second technical replicate analyses corresponding to those of FIG. 1 showing repeat measurements of dry-weight concentrations of the nine essential elements (A-I) in four brain regions compared between control (red) and T2D (green) human post-mortem tissue. Data are means±95% CI. Within the MN, a single outlier from Mg and Cu was removed from the plot for clarity of presentation: this did not alter the results or conclusions. Non-standard abbreviations: FC: Frontal cortex; TC: Temporal cortex; HP: Hippocampus; MN: Meninges.

FIG. 3 shows two-dimensional PCA and PLS-DA plots with VIP scores for human dry-weight hippocampal postmortem tissue. Data represents A) PCA and B) PLS-DA plots for AD (red; n=9), control (green; n=14), and T2D (blue; n=6) hippocampal tissue. The coloured ellipses for the PCA and PLS-DA plots represent 95% confidence regions. In the PCA plot, the first principal component (PCI) represents 48.3% of the total variance whereas the second (PC2) contributes 19.3%. The two cases (AD vs. T2D) demonstrate almost complete separation whereas a substantial overlap is apparent between T2D and controls in both PCA and PLS-DA plots. The VIP score plots (bottom) show the relative contribution of each metal to the variance between AD, T2D, and controls in C) component 1 and D) component 2. A larger VIP score indicates a greater contribution to the separation of groups. The colored boxes to the right indicate whether metal concentrations are increased (red) or decreased (green) in the affected group. For both components, Cu, Na, and Mn achieved the top three highest VIP scores, with Na in both components achieving the highest VIP score. Non-standard abbreviations: AD: Alzheimer's disease; Con: Controls; PCA: Principal component analysis; PC: Principal component; PLS-DA: Partial least square-discriminant analysis; T2D: Type-2 diabetes; VIP: Variable importance for projection.

FIG. 4 shows a scree plot for Dry-weight hippocampal post-Morten tissue. Data represent a scree plot showing the individual variance explain to the top five dimensions after hippocampal PCA.

FIG. 5 shows two-dimensional PCA and PLS-DA plots for human dry-weight T2D temporal cortex and AD temporal gyms post-mortem tissue. Data represents FIG. 5(A) PCA and FIG. 5(B) PLS-DA plots for AD (red; n=9; mineral temporal gyms), control (green; n=13; temporal cortex/middle temporal gyrus), and T2D (blue; n=6: temporal cortex). The colored ellipses for the PCA and PLS-DA plots represent 95% confidence regions. And the PCA plot, the first principal component (PC1) represents 41.2% of the total variance whereas the second (PC2) contributes 24.5%. The two cases (AD vs. T2D) show no separation in the PCA plot, whereas, in the PLS-DA plot, the separation is visible due to the inclusion of supervised modeling

FIG. 6 shows two-dimensional PCA and PLS-DA plots for human dry-weight frontal cortex and meningeal post-mortem tissue. Data represents PCA (FIG. 6A & FIG. 6C) and PLS-DA (FIG. 6B & FIG. 6C) plots for controls (red; n=6) and T2D (green; n=6) frontal cortex (FIG. 6A & FIG. 6B) and meningeal (FIG. 6C & FIG. 6D) tissue. The colored ellipses for the PCA and PLS-DA plots represent 95% confident regions. No separation was visible in any of the multivariate plots for both frontal cortex and meningeal post-mortem tissue.

### DETAILED DESCRIPTION

There is an epidemic of both diabetes and dementia among older adults. The risk for dementia is increased in patients with diabetes, and patients with dementia and diabetes appear to be at greater risk for severe hypoglycemia. The precise factors that contribute to the increased risk for dementia in older adults with diabetes are not known. Neurobiological alterations seen in the aging hippocampus—including increased oxidative stress and neuroinflammation, altered intracellular signaling and gene expression, as well as reduced neurogenesis and synaptic plasticity—are thought to be associated with age-related cognitive decline. Diabetes, including type 2 diabetes mellitus, for example, has been shown to increase the risk for cognitive decline and dementia, such as in Alzheimer's disease and vascular dementia.

The deleterious effects of diabetes mellitus on the retinal, renal, cardiovascular, and peripheral nervous systems are widely acknowledged. Less attention has been given to the effect of diabetes on cognitive function. Both type 1 and type 2 diabetes mellitus have been associated with reduced performance on numerous domains of cognitive function. The exact pathophysiology of cognitive dysfunction in diabetes is not completely understood, but it is believed that hyperglycemia, vascular disease, hypoglycemia, and insulin resistance may play significant roles. Modalities to study the effect of diabetes on the brain have evolved over the years, including neurocognitive testing, evoked response potentials, and magnetic resonance imaging, and are useful in conjunction with the methods of the invention both before and during treatment.

A recent meta-analysis included 33 studies examining cognitive function in adult subjects with diabetes mellitus. Kodl and Seaquist, Cognitive Dysfunction and Diabetes, Endocr Rev. 2008 June; 29(4): 494-511. It found that there were significant reductions in overall cognition, fluid and crystallized intelligence, speed of information processing, psychomotor efficiency, visual and sustained attention, mental flexibility, and visual perception in subjects with type 1 diabetes compared with controls. There was no difference in memory, motor speed, selective attention, and language. (see Table 1 in Kodl and Seaquist.)

Kodl and Seaquist report in their article that patients with type 2 diabetes mellitus have also been found to have cognitive impairment (id.; see Table 2 in Kodl and Seaquist). The authors provide citations to note that type 2 diabetes has been associated with decreases in psychomotor speed, frontal lobe/executive function, verbal memory, processing speed, complex motor functioning, working memory, immediate recall, delayed recall, verbal fluency, visual retention, and attention. The authors refer to a study by Sinclair et al. (2000 Cognitive dysfunction in older subjects with diabetes mellitus: impact on diabetes self-management and use of care services. All Wales Research into Elderly (AWARE) Study. Diabetes Res Clin Pract 50:203-212), who found that subjects with mini-mental status exam scores less than 23 fared worse on measures of self-care and ability to perform activities of daily living, and also displayed an increased need for personal care and increased rates of hospitalization when compared with controls. Type 2 patients also have an increased incidence of Alzheimer's disease and increased incidence of vascular dementia. Recently, Bruce et al. found that 17.5% of elderly patients with type 2 diabetes had moderate to severe deficits in activities of daily living, 11.3% had cognitive impairment, and 14.2% had depression (2003 Cognitive impairment, physical disability and depressive symptoms in older diabetic patients: the Fremantle Cognition in Diabetes Study. Diabetes Res Clin Pract 61:59-67). See also Zillox L., et al., Diabetes and Cognitive Impairment, Curr Diab Rep. 2016 September; 16(9): 87.

Type 2 diabetes is characterized by chronic hyperglycemia and a propensity for glucose-mediated damage in numerous organs; impaired copper (Cu) homeostasis; elevated rates of impaired cognitive function and dementia; and epidemiological associations with sporadic Alzheimer's disease (sAD). By contrast, sAD exhibits widespread, progressive age-related cerebral neurodegeneration and dementia; pervasive elevation of brain glucose without significant hyperglycaemia; as well as pervasive cerebral-Cu deficiency.

The study in the below Example describes methods to determine whether sAD-like brain-metal perturbations occur in T2D, where levels of nine essential metals were measured from four brain regions in cases with short post-mortem delay (n=6) and matched controls (n=6) and compared with case-control data from a study of sAD (n=9/group) performed using equivalent methodology. Inter-group differences in hippocampal data using both supervised and unsupervised multivariate-statistical methods were contrasted. To determine whether sAD-like brain-metal perturbations occur in type 2 diabetes, levels of nine essential metals from four brain regions in cases with short post-mortem delay (n=6) and matched controls (n=6) were measured and compared with case-control data from a study of sAD (n=9/group) performed using equivalent methodology. Inter-group differences in hippocampal data were contrasted using both supervised and unsupervised multivariate-statistical methods.

Unexpectedly, it was discovered that hippocampal-Cu levels were markedly elevated in T2D (P=0.005 and 0.007 in sequential technical-replicate experiments), approximating literature values in Wilson's disease (WD) brain, whereas, contrastingly, hippocampal-Cu values in sAD were severely deficient. Multivariate analysis identified marked differences in corresponding metal-related patterns between hippocampal case-control datasets from type 2 diabetes and sAD.

In other words, brain hippocampal copper is markedly elevated in type 2 diabetes, while hippocampal copper values in the brains of sporadic Alzheimer's disease patients are severely deficient. It is understood that the failure to remove excess copper in the WD brain can lead to irreversible brain damage.

The hippocampus is often impacted early in the development of both type 2 diabetes- and sAD-evoked neurodegeneration. As show in the below Example, hippocampal-Cu levels were markedly elevated in type 2 diabetes cases whereas, contrastingly, hippocampal-Cu was deficient in sAD, consistent with the severe, widespread brain-Cu deficiency reported therein. Mechanisms of altered hippocampal-Cu thus differ between type 2 diabetes and sAD. Elevated hippocampal Cu will contribute to the pathogenesis of cerebral neurodegeneration and cognitive impairment in type 2 diabetes, consistent with known adverse impacts of similarly elevated cerebral Cu in WD. Therapeutic brain-copper-lowering approaches mirroring those currently employed in WD will be useful in patients with type 2 diabetes who show impaired cognitive function or signs of cerebral neurodegeneration, etc., as described herein.

Referring to the known adverse impacts of elevated cerebral copper in WD, elevated hippocampal Cu levels are consistent with their contribution to the pathogenesis of cerebral neurodegeneration and cognitive impairment in type 2 diabetes. Therapeutic hippocampal and brain copper-lowering approaches will be useful in patients with type 2 diabetes who show impaired cognitive function or signs of cerebral neurodegeneration, are at risk therefor, or are positive after testing for increased hippocampal and/or brain copper.

The below Example shows that elevation in hippocampal Cu levels was the only substantive perturbation in essential-metal homeostasis detected in the brain of patients with type 2 diabetes. By contrast, there were no substantive case-control differences in tissues from the frontal or temporal cortices, or meninges.

To further determine the significance of these findings, additional analyses were conducted to contrast the observations in this case-control study of type 2 diabetes with individual patient measurements derived from a directly comparable case-control study of sAD wherein brain-metal levels were determined by the same methods as those employed here for the type 2 diabetes and control subjects. Unexpectedly and in direct contrast to the findings in type 2 diabetes, hippocampal-Cu levels in sAD were substantively lower than those in the matched controls. Therefore, the measured pattern of Cu dysregulation in sAD (that is, hippocampal-Cu deficiency) contrasted strongly with that in type 2 diabetes (hippocampal-Cu elevation). Thus, hippocampal Cu levels are elevated in type 2 diabetes compared to control values, whereas in sAD they are lowered compared with controls. Furthermore, the Cu perturbations in sAD are widespread, where those in type 2 diabetes may be more restricted. It can be concluded that the processes that give rise to or cause more limited defective Cu homeostasis in type 2 diabetes differ fundamentally from those that cause widespread brain-Cu deficiency in sAD.

Cu is the third most abundant transition metal in the brain (after Zn and Fe), where it performs essential roles in many processes including cellular respiration, the regulation of Fe metabolism, and antioxidant pathways. Defective Cu regulation is known to play central roles in the pathogenesis of two genetic disorders: WD, which is characterized by toxic Cu overload in the liver, eye and brain, and Menkes' disease where, on the contrary, brain damage is caused by brain-Cu deficiency. Both of these diseases can cause severe neurodegeneration unless normal brain-Cu levels are restored by prompt pharmacological intervention following diagnosis. Here, it was ascertained that the increased Cu levels measured in the T2D hippocampus approximated fold-changes in regions adjacent to the hippocampus, as reported for WD (Table 3).

Our root cause (RCA) analysis revealed a virtually complete separation between signals from sAD and type 2 diabetes, whereas type 2 diabetes and control hippocampal tissue showed substantive overlap. This contrasts with the parallel findings in the other type 2 diabetes brain regions studied, where case and control values substantively overlapped. To further examine discrimination between cases and controls, PLS-DA (partial least squares-discriminant analysis) modelling was employed, which displayed similar cluster separation to the RCA analysis. These findings further clarify the contrasting patterns of brain-metal dyshomeostasis between type 2 diabetes and sAD. Based on the PLS-DA modelling, VIP scores revealed that Na, Mn, Cu, and Fe each had scores of >1, showing that these metals provide a reliable discriminant for cluster separation. Although Na achieved the top VIP score in both components, Cu was ranked third and second highest for VIP scores in component 1 and 2, respectively. This shows that Cu is one of the key variables responsible for the separation of AD and type 2 diabetes clustering and supports the conclusion that Cu perturbations play a key role in the neuropathogenesis of type 2 diabetes. Mirroring our initial ICP-MS findings, the identification of metal dysregulation using the same multivariate analysis was not apparent between type 2 diabetes and controls in the remaining brain regions. Despite the previous observations of shared pathophysiological traits between AD and type 2 diabetes (Chatterjee S, Mudher A. Alzheimer's disease and type 2 diabetes: A critical assessment of the shared pathological traits. Front Neurosci 2018 Jun. 8; 12:383) these findings support the likelihood of contrasting Cu-related neurodegenerative processes in these two diseases.

To probe the possible impacts of the sample sizes in the present study (T2D vs con; n=6/6), post-hoc statistical power tests for hippocampal tissue were conducted to confirm the lack of a type II error. Across the two technical replicates, Cu had the highest power level (>0.90; see Table 7 in the below Example) whereas all the other metals had power levels <0.80. Furthermore, a priori analysis identified that only Cu had a desired sample-size limit (n=10) below that which was used in this study. These power levels are considered when interpreting the measured differences in other hippocampal-metal levels. A recent paper reported that brain-metal levels are unaffected by PMD lengths of up to 72 h. Thus, it is also concluded that the PMDs in the present study did not significantly affect brain-metal levels. See Scholefield M, et al. Evidence that levels of nine essential metals in postmortem human-Alzheimer's-brain and ex vivo rat-brain tissues are unaffected by differences in postmortem delay, age, disease staging, and brain bank location. Metallomics 2020; 12: 952-62.

In sum, the study in the Example provides robust evidence for increased hippocampal-Cu levels in cases of type 2 diabetes, whereas hippocampal Cu was correspondingly decreased in sAD. This is the first study to report multiregional metal concentrations in the type 2 diabetes brain using metallomic methods, which allow quantitative measurements of all essential metals simultaneously in a manner not accessible to other methodologies such as MRI. The Cu fold-change in type 2 diabetes approximated those reported in WD, wherein neurodegeneration is due to Cu toxicity. Substantively contrasting patterns of brain-metal levels were identified using PCA and PLS-DA among type 2 diabetes, sAD, and controls. Cu had among the highest VIP scores indicating that it is a key discriminant factor responsible for the separation between sAD and type 2 diabetes clustering here. Taken together, these findings demonstrate the existence of contrasting neurodegenerative processes in type 2 diabetes and sAD.

Therapeutic copper-lowering and copper-normalizing approaches are disclosed and claimed for treating patients with diabetes who show or are at risk for impaired cognitive function, cognitive decline and/or dementia or show signs of cerebral neurodegeneration. The methods of the invention are also useful for the prophylactic treatment or prevention of impaired cognitive function, cognitive decline and/or dementia, etc. in patients with diabetes. Patients include those with diabetes, for example, type 2 diabetes, and/or vascular dementia, with or without diabetes. The methods of the invention are also useful for reducing chelatable copper in these subjects to treat or prevent these conditions.

In some embodiments, therapeutic copper-lowering and copper-normalizing approaches are disclosed and claimed for treating patients (both actively or prophylactically) having or suspected of having or at risk for fronto-temporal dementia (FTD). In some embodiments of the methods, one or more symptoms of fronto-temporal dementia is/are reduced or alleviated. In some method embodiments, one or more symptoms of fronto-temporal dementia is/are substantially eliminated.

In some embodiments, therapeutic copper-lowering and copper-normalizing approaches are disclosed and claimed for treating patients (both actively or prophylactically) having or suspected of having or at risk for fronto-temporal lobar dementia (FTLD). In some embodiments of the methods, one or more symptoms of fronto-temporal lobar dementia is/are reduced or alleviated. In some method embodiments, one or more symptoms of fronto-temporal lobar dementia is/are substantially eliminated.

In some embodiments, therapeutic copper-lowering and copper-normalizing approaches are disclosed and claimed for treating patients (both actively or prophylactically) having or suspected of having or at risk for amyotrophic lateral sclerosis (ALS) and/or accompanying motor neuron disease (MND) dementia. In some embodiments of the methods, one or more symptoms of ALS and/or MND is/are reduced or alleviated. In some method embodiments, one or more symptoms of ALS and/or MND is/are substantially eliminated.

In some embodiments, therapeutic copper-lowering and copper-normalizing approaches are disclosed and claimed for treating patients (both actively or prophylactically) having or suspected of having or at risk for dementia with Lewy bodies (DLB). In some embodiments of the methods, one or more symptoms of DLB is/are reduced or alleviated. In some method embodiments, one or more symptoms of DLB is/are substantially eliminated.

In some embodiments, therapeutic copper-lowering and copper-normalizing approaches are disclosed and claimed for treating patients (both actively or prophylactically) having or suspected of having or at risk for multiple sclerosis (MS). In some embodiments of the methods, one or more symptoms of MS is/are reduced or alleviated. In some method embodiments, one or more symptoms of MS is/are substantially eliminated.

In some embodiments, methods of treating FTD, FTLD, ALS, MND, DLB and/or MS comprises or consists essentially of administering to the subject a pharmaceutical composition comprising a compound capable of normalizing copper metabolism. In some embodiments, the compound capable of reducing or lowering copper values, copper levels or total copper or capable of normalizing copper values, copper levels, total copper or copper metabolism is a copper chelator or other copper binding or copper removing agent. Other embodiments of these methods for the treatment of FTD, FTLD, ALS, MND, DLB and/or MS, and the agents, compounds, compositions, and procedures useful in these methods, are described herein, including for example copper antagonists, copper chelating agents, and compounds capable of normalizing copper values useful therein and in the treatment thereof.

### Definitions

Copper(II) referred to herein is also known as Cu(II) or Cu⁺² or copper⁺², or as “cupric” (the copper⁺² cation). Copper(I) referred to herein is also known as Cu(I) or Cu⁺¹ or copper⁺, or as “cuprous” (the copper⁺¹ cation).

The term “chelatable copper” as used herein includes copper in any of its chelatable forms including different oxidation states such as copper(I) and copper(II). Accordingly, the term “copper values” (for example, elemental, salts, etc.) means copper in any appropriate form in the body available for such chelation (for example, in the hippocampus) and/or capable of being lowered or removed by other means. Certain methods and compositions of the invention may be used to bind chelatable copper, for example, chelatable copper(II) to reduce hippocampal copper while maintaining normal or near-normal copper values (e.g., within about 70-110% of normal, for example, 75-105% of normal, 80-100% of normal, or other copper values amount not detrimental to the subject).

The term “pharmaceutical formulation” refers to a preparation which is in such form as to permit the biological activity of an active ingredient contained therein to be effective, and which does not contain additional components that are unacceptably toxic to a subject to which the formulation would be administered. Pharmaceutical formulations of the invention useful for lowering hippocampal copper in diabetes comprise or consist essentially of one or more copper antagonists, such as, for example, one or more copper-depriving agents, e.g., one or more copper chelators or binding agents (alone or together with other therapeutic agents), one or more copper sequestering agents and/or one or more copper removing agents. Pharmaceutical compositions as used herein are “pharmaceutical formulations.” In some embodiments, pharmaceutical formulations or compositions of the invention comprise a trientine, e.g., triethylenetetramine disuccinate, and a pharmaceutically acceptable carrier. In some embodiments, pharmaceutical formulations or compositions are specifically designed for administration to people with diabetes.

Copper antagonists, including copper chelating agents, copper sequestering agents, copper depriving agents, copper lowering agents, copper removing agents, alone or together with other therapeutic agents, including anti-dementia medications, therapeutic agents selected from an anti-inflammatory agent, an agent for treating cardiovascular disease, an agent for treating hypertension, an agent for treating kidney disease, an agent for treating depression, and an agent for treating type 2 diabetes and/or dementia, or any of the other disorders disclosed herein, may be administered alone or in combination with one or more additional ingredients and may be formulated into pharmaceutical compositions including one or more pharmaceutically acceptable excipients, diluents and/or carriers.

By “pharmaceutically acceptable” it is meant, for example, a carrier, diluent or excipient that is compatible with the other ingredients of the formulation and generally safe for administration to a recipient thereof or that does not cause an undesired adverse physical reaction upon administration. A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative. Pharmaceutically acceptable diluents, carriers and/or excipients include substances that are useful in preparing a pharmaceutical composition, may be co-administered with compounds described herein while allowing them to perform its intended functions, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of the invention. However, by way of example, diluents, carriers and/or excipients include solutions, solvents, dispersion media, delay agents, polymeric and lipidic agents, microspheres, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration, for example, and vehicles such as liposomes being also suitable for administration of the agents of the invention.

“Copper chelating agents” bind or modify copper, including those that selectively bind to or modify copper(I) or copper(II) values and are used to reduce or normalize blood and/or tissue copper levels and to prevent unwanted copper accumulation. Copper chelating agents include prodrugs thereof. Other agents that normalize copper values, and other agents that selectively bind to or modify copper(II), whether now known or later developed, are included within this definition.

A “copper antagonist” includes “copper sequestering agents” and “copper-depriving agents” and “copper lowering agents” and is an agent that can reduce, bind to and/or suppress the ability of copper in any or all of its various forms, for example, as copper atoms or copper ions (including copper(II) and/or copper(I)), to interact in any chemical or physical reactions that it could otherwise do, including in the hippocampus. Copper-depriving agents include chelators, agents that reduce total copper amounts, agents that reduce copper values, agents that reduce the amount of intracellular copper, including those described herein. Copper-depriving agents also include copper-modifying agents, i.e., agents used to reduce hippocampal copper by modifying copper content in the body, including intracellular content, or by modifying copper availability. It is understood that copper is an essential intracellular nutrient, and thus the invention includes methods to reduce intracellular copper content while maintaining safe patient copper levels. Copper-depriving agents include copper-removing agents, i.e., agents that remove copper from the body and/or from inside cells. A “copper removing agent” is a compound that can bind selectively to and remove copper from its binding sites in the body in the form of a complex, whereby the complexed Cu ions are removed preferentially from the tissues via the blood plasma into the urine or the feces and thence from the body. The “copper removing agent” compound may be selective for Cu(I), for Cu(II), or similarly or equivalently for Cu(I) and Cu(II).

A “compound capable of normalizing copper values” is a compound that binds selectively to and removes copper (Cu) from its binding sites in the tissues of a mammal such as a human, wherein the Cu removed is usually in the form of a complex, and through which binding process the Cu content of the body is restored to levels not significantly different from those in comparable physiologically normal individuals.

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of compounds, compositions and methods of the invention to reduce copper levels, to reduce total copper, to reduce copper values, to lower copper, preferably copper(II), and/or to chelate copper, preferably copper(II). The basic and novel characteristics of the inventions also include the ability of compounds, compositions and methods of the invention to provide a clinically relevant change in impaired cognitive function in a subject with diabetes or vascular dementia by lowering or normalizing copper, copper values and/or total copper and, thus, hippocampal copper. In another aspect of one of the methods of the inventions, the basic and novel characteristics of other compositions and methods of the invention include the ability to prevent or reduce, at least in part, impaired cognitive function, cognitive decline, and/or dementia or show signs of cerebral neurodegeneration in a subject with diabetes, for example, type 2 diabetes. These concepts incorporate, for example, the ability to reduce or eliminate the deleterious effects of excessive hippocampal copper. Some signs of impaired cognitive function, cognitive decline, dementia, and cerebral neurodegeneration in a subject with diabetes are described herein, but are not limited to those particular signs but include such signs known in the art or later identified.

As used herein, the term “subject” or the like, including “individual,” and “patient”, all of which may be used interchangeably herein, refers to any mammal, including humans. The preferred mammal herein is a human, including adults, children, including those with diabetes or vascular dementia, by way of example. In certain embodiments, the subject, individual or patient is a human. In some embodiments, the subject has hippocampal and/or amygdala atrophy or another hippocampal structural defect. In some embodiments, the hippocampal and/or amygdala atrophy or other hippocampal structural defect is visualized in subjects with diabetes using MRI or other imaging devices. In some embodiments, structural correlates of diabetes-related cognitive impairment in subjects are assessed with brain magnetic resonance imaging (MRI) prior to and/or during treatment. In some embodiments, in evaluating cognitive impairment in patients with diabetes for treatment as described herein, e.g., in patients with type 2 diabetes, MRI is used to examine or confirm the presence of normal or abnormal cerebral structure before and/or during treatment. In some embodiments, in patients with type 2 diabetes, for example, white matter hyperintensities are correlated with reduced performance on tests of attention, executive function, information processing speed, and memory and provide a structural basis for treatment with compounds of the invention. In some embodiments, MRI is used to examine or confirm the presence of white matter hyperintensities before and/or during treatment. In some embodiments, MRI is used to demonstrate or confirm that subjects with diabetes, e.g., type 2 diabetes, have lacunar infarction(s), hippocampal atrophy and/or amygdala atrophy or other hippocampal defects, including hippocampal atrophy patterns, prior to treatment with compounds of the invention. In some embodiments, MRI is used to evaluate subjects with diabetes, e.g., type 2 diabetes, for hippocampal and/or amygdala atrophy or other hippocampal defects, including hippocampal atrophy patterns, during treatment with compounds of the invention. In some embodiments, MRI is used to assess hippocampal and/or amygdala atrophy in subjects with type 2 diabetes prior to, during and/or after treatment according to methods of the invention.

As used herein, “mammal” has its usual meaning and includes primates (e.g., humans and nonhumans primates), experimental animals (e.g., rodents such as mice and rats), farm animals (such as cows, hogs, minks, sheep and horses), and domestic animals (such as dogs and cats).

As used herein the terms “subjecting the patient” or “administering to” includes any active or passive mode of ensuring the in vivo presence of a compound for lowering or normalizing copper, copper values, total copper, etc., including levels of hippocampal copper, e.g., triethylenetetramine disuccinate. Preferably the mode of administration is oral. However, all other modes of administration (particularly parenteral, e.g., intravenous, intramuscular, CNS, etc.) are contemplated. Also contemplated are nasal administration to bypass the blood-brain barrier, administration of compounds of the invention with blood-brain barrier penetration enhancers, and cerebrospinal fluid delivery.

The term “treating impaired cognitive function” or the like, refers to preventing, slowing, reducing, lowering, decreasing, stopping and/or reversing an impaired cognitive function in a subject, or one or more symptoms thereof, including, for example, one or more of reductions in overall cognition, fluid and crystallized intelligence, speed of information processing, psychomotor efficiency, visual and sustained attention, mental flexibility, and/or visual perception. The term “treating impaired cognitive function” or the like, may also refers to preventing, slowing, reducing, lowering, decreasing, stopping and/or reversing a impaired cognitive function in a subject, or one or more symptoms thereof, including, for example, one or more of impairment in memory (verbal memory, visual retention, working memory, immediate recall, and/or delayed recall, etc.), psychomotor speed and frontal lobe/executive function, processing speed, complex motor function, verbal fluency and/or attention. In some embodiments, the cognitive impairment prevents an individual from concentrating, recalling memories, and/or leads to mental fatigue. The compounds and methods of treatment described herein may be used to treat impaired cognitive function in diseases, disorders or conditions characterized by excess or unwanted levels of copper in the hippocampus. The compounds and methods of treatment may be used to treat impaired cognitive function, cognitive decline, and/or dementia or signs of cerebral neurodegeneration in a subject with diabetes, including for example, type 2 diabetes. In some embodiments, the subject being treated has type 1 diabetes and one or more of the cognitive domains negatively affected by cognitive impairment in the subject is a reduction in overall cognition, fluid and crystallized intelligence, speed of information processing, psychomotor efficiency, visual and sustained attention, mental flexibility, and/or visual perception. In some embodiments, the subject being treated has type 2 diabetes and one or more of the cognitive domains negatively affected by cognitive impairment in the subject is memory (verbal memory, visual retention, working memory, immediate recall, delayed recall), psychomotor speed and frontal lobe/executive function, processing speed, complex motor function, verbal fluency and/or attention. In some embodiments, the cognitive impairment that is treated or prevents is one that prevents an individual with diabetes, for example, type 2 diabetes, from concentrating, recalling memories, and/or leads to mental fatigue.

“Treating copper excess” refers to preventing, slowing, reducing, lowering, decreasing, stopping and/or reversing, in whole or in part, pathological, excess or unwanted copper in the hippocampus of a subject, and/or to treating one or more symptoms of excess or unwanted copper. The compounds and methods of treatment described herein may be used to treat copper excess. The compounds and methods of treatment described herein may be used to remove or lower undesired levels or amounts of chelatable copper. Subjects will be evaluated prior to and/or during treatment and copper in the urine (or another source) will be measured and/or slowing or reversing of the shrinkage of the hippocampus demonstrated by one or more imaging methods, e.g., by MRI. In some embodiments, excess copper in a subject can be measured by methods such as mass spectrometry of a tissue sample, CSF (cerebral spinal fluid) aspirate, and/or urine copper output. In some embodiments, treating copper excess can be measured by the slowing or reversing of the shrinkage of the hippocampus by known imaging methods (e.g. MRI—magnetic resonance imaging) of a subject undergoing treatment.

The term “preventing” means preventing in whole or in part or ameliorating or controlling. Thus, preventing impaired cognitive function, cognitive decline, and/or dementia or signs of cerebral neurodegeneration means preventing in whole or in part, or ameliorating or controlling one or more symptoms of one or more of these conditions. The compounds and methods of treatment described herein may be used to prevent copper excess leading to impaired cognitive function, cognitive decline, and/or dementia or signs of cerebral neurodegeneration in diabetes, including, for example, in type 2 diabetes.

As used herein, the terms “effective amount” or “therapeutically effective amount” refer to an amount of a compound useful for treating hippocampal copper excess. The terms “effective amount” or “therapeutically effective amount” are also used to refer to an amount of a copper antagonist compound for treating and/or preventing impaired cognitive function, cognitive decline, and/or dementia or signs of cerebral neurodegeneration in diabetes, including, for example, in type 2 diabetes. Triethylenetetramine disuccinate is one such compound. Exemplary effective amounts of this compound are described herein, and include doses in the range of from about 2400 mg per day to about 3000 mg per day comprising or consisting essentially of fixed doses of triethylenetetramine disuccinate. In one aspect, the effective amount of triethylenetetramine disuccinate is at least about 95% pure, at least about 99% pure, or 100% pure. In another aspect, the effective amount of triethylenetetramine disuccinate is a crystalline form of triethylenetetramine disuccinate. In yet another aspect of this method, the effective amount of triethylenetetramine disuccinate is a triethylenetetramine disuccinate anhydrate. In still another aspect of the method the effective amount of the triethylenetetramine disuccinate is in the form of a fixed dose tablet or capsule. In one preferred embodiment, the effective fixed dose of triethylenetetramine disuccinate is about 350 mg, 400 mg, about 500 mg, about 600 mg or about 700 mg.

Thus, in one aspect, “effective amount” refers to an amount effective, at dosages and for periods of time necessary, to achieve the desired therapeutic or prophylactic result. For example, and not by way of limitation, an “effective amount” can refer to an amount of a copper antagonist, such as a copper sequestering agent or copper-depriving agent (including those disclosed herein, including, e.g., copper chelators such as triethylenetetramine disuccinate) that is able to treat the signs and/or symptoms of impaired cognitive function, cognitive decline, and/or dementia or signs of cerebral neurodegeneration in people with diabetes by reducing or lowering hippocampal copper or hippocampal copper le. Advantageously, these compounds and methods will also reduce copper excess in other areas of the brain. In one embodiment, the effectiveness of the amount is evaluated by determining the response of the subject and/or the amount copper in the urine or plasma of a subject following the dosing of a copper antagonist as disclosed herein. Preferably, the effective amount maintains normal copper levels, or maintains a subject's copper levels within at least about 70% of normal, preferably within at least about 80% of normal, within at least about 90% of normal, within or within other levels described herein. In one embodiment, these are urinary copper levels. In other embodiments, serum copper is lowered not more than about 5-10%, i.e., serum copper is maintained within about 90-95% of normal and serves as a safety variable.

As used herein, “prophylactically effective amount” refers to an amount effective, at dosages and for periods of time necessary, to achieve a desired prophylactic result. Typically, but not necessarily, since a prophylactic dose of a copper antagonist is used in subjects prior to or at an earlier stage of impaired cognitive function, cognitive decline, and/or dementia or signs of cerebral neurodegeneration, the prophylactically effective amount may be less than the therapeutically effective amount. Prophylactic doses may also serve as maintenance doses once impaired cognitive function, cognitive decline, and/or dementia or signs of cerebral neurodegeneration have been brought under control with, for example, an initial, bolus or loading dose or doses, all as described herein, for example.

As used herein, the terms “treatment” or “treating” of the signs and/or symptoms of impaired cognitive function, cognitive decline, and/or dementia or signs of cerebral neurodegeneration in a mammal, means, (i) preventing the condition or disease, that is, avoiding one or more clinical symptoms of impaired cognitive function, cognitive decline, and/or dementia or signs of cerebral neurodegeneration; (ii) inhibiting impaired cognitive function, cognitive decline, and/or dementia or signs of cerebral neurodegeneration that is, arresting the development or progression of one or more clinical symptoms of impaired cognitive function cognitive decline, and/or dementia or signs of cerebral neurodegeneration; and/or (iii) relieving the impaired cognitive function, cognitive decline, and/or dementia or signs of cerebral neurodegeneration, that is, causing the regression of one or more clinical symptoms, including one or more of the symptoms described herein. Thus, “treatment” (and grammatical variations thereof such as “treat” or “treating”) normally refers to clinical intervention in an attempt to alter the natural course of the individual, tissue or cell being treated, and can be performed either for prophylaxis or during the course of clinical pathology. The term does not necessarily imply that a subject is treated until total recovery. Accordingly, “treatment” includes reducing, lowering, alleviating or ameliorating the symptoms or severity of impaired cognitive function, cognitive decline, and/or dementia or signs of cerebral neurodegeneration, or preventing or otherwise reducing the risk of developing impaired cognitive function, cognitive decline, and/or dementia or signs of cerebral neurodegeneration. It may also include maintaining or promoting a complete or partial state of remission of impaired cognitive function, cognitive decline, and/or dementia or signs of cerebral neurodegeneration. The copper antagonist compounds described herein, including, for example, triethylenetetramine disuccinate, are used for treatment.

As used herein “associated with” simply means both circumstances exist and should not be interpreted as meaning one necessarily is causally linked to the other.

Structural correlates of diabetes-related cognitive impairment can be assessed with brain magnetic resonance imaging (MRI) prior to and/or during treatment as described and claimed herein. See Jongen C, Biessels G J: Structural brain imaging in diabetes: a methodological perspective. Eur J Pharmacol 2008;585: 208-218.

In some embodiments, as noted, in evaluating cognitive impairment in patients with diabetes for (or during) treatment as described herein, e.g., type 2 diabetes, magnetic resonance imaging (MRI) is used to examine or confirm the presence of normal or abnormal cerebral structure before and/or during treatment. In some embodiments, in patients with type 2 diabetes, for example, white matter hyperintensities are correlate with reduced performance on tests of attention, executive function, information processing speed, and memory and provide a structural basis for treatment with compounds of the invention. In some embodiments, as noted, MRI is used to demonstrate or confirm that subjects with type 2 diabetes have hippocampal and/or amygdala atrophy before and/or during treatment with compounds of the invention. In some embodiments, MRI is used to assess hippocampal and/or amygdala normality and/or signs of atrophy or shrinkage in subjects with diabetes prior to and/or during treatment with compounds of the invention. The middle temporal gyrus and/or entorhinal cortex may also be evaluated for abnormalities prior to and/or restoration during treatment. In some embodiments, the subject has type 2 diabetes. The hippocampus and amygdala are responsible for such functions as memory and behavior and, interestingly, are also found to be atrophied in Alzheimer's patients.

The invention provides methods for treating or preventing impaired cognitive function, cognitive decline, and/or dementia or signs of cerebral neurodegeneration in a subject with diabetes mellitus, comprising administering to the subject a pharmaceutical composition comprising a compound capable of reducing, lowering or normalizing hippocampal copper levels or amounts. In some embodiments, the compound is capable of treating copper excess. In some embodiments, the compound is a copper antagonist. In some embodiments, the copper antagonist is a copper chelator or other copper binding or removing or sequestering agent. In some embodiments, the copper chelator or other copper binding agent is a trientine. In some embodiments, the trientine is triethylenetetramine disuccinate. In other embodiments, the trientine is triethylenetetramine dihydrochloride or tetrahydrochloride. In other embodiments, the compound is another compound effective to reduce copper, lower total copper or lower the copper values content in a subject and thus hippocampal copper as set forth herein. In other embodiments, the compound is another compound effective to reduce copper, lower total copper or lower the copper values content in a subject and thus hippocampal copper is one that is now known or later identified in the art.

The invention also provides methods for treating or preventing cognitive and/or memory impairment in subjects with diabetes. In some embodiments, the subject has type 2 diabetes. In other embodiments, the subject has type 1 diabetes. In some embodiments, the subject has type 3 diabetes. In some embodiments, the subject has type 4 diabetes.

In some embodiments, invention provides methods for treating a subject with a copper antagonist who has diabetes, for example, type 1 diabetes, and a reduction in one or more of overall cognition, fluid and crystallized intelligence, speed of information processing, psychomotor efficiency, visual and sustained attention, mental flexibility, and/or visual perception by administering one or more compounds or composition of the invention in order to improve one or more of overall cognition, fluid and crystallized intelligence, speed of information processing, psychomotor efficiency, visual and sustained attention, mental flexibility, and/or visual perception in the subject.

In some embodiments, invention provides methods for treating a subject with diabetes, for example, type 2 diabetes, and one or more negatively affected cognitive domains, including memory (verbal memory, visual retention, working memory, and/or immediate recall and/or delayed recall), psychomotor speed and frontal lobe/executive function, processing speed, complex motor function, verbal fluency and/or attention by administering one or more compounds or composition of the invention in order to improve one or more of these or another negatively affected cognitive domains in the subject. In some embodiments, the cognitive impairment to be treated to prevented is one that prevents an individual with diabetes, for example, type 2 diabetes, from concentrating, recalling memories, and/or leads to mental fatigue.

In other embodiments, the invention relates to methods of treating or preventing impaired cognitive function or cognitive decline in a subject with vascular dementia, also sometimes referred to as vascular cognitive impairment, or VCI, comprising administering to the subject a pharmaceutical composition comprising a copper antagonist, including compounds capable of reducing, lowering and/or normalizing copper metabolism. In some embodiments, the subject may or may not have diabetes mellitus. In some embodiments, the copper antagonist compound is a copper chelator or other copper binding agent. In some embodiments, the subject with vascular dementia or VCI has problems with reasoning, planning, judgment, memory and other thought processes, and the compounds, compositions and methods of the invention reduce and alleviate these problems, in whole or in part.

In some embodiments of the methods of the invention, the compound capable of normalizing copper metabolism is capable of lowering or alleviating elevated copper levels and elevating reduced copper levels in a subject. In some embodiments, the compound capable of lowering elevated copper levels and elevating lowered copper levels in a subject is a trientine. In some embodiments, the trientine is triethylenetetramine disuccinate.

In some embodiments of the methods of the invention, the copper antagonist is a copper binding compound that binds copper²⁺. In some embodiments, the copper binding compound is a copper chelator. In some embodiments, the copper chelator chelates copper²⁺. In some embodiments of the methods of the invention, the copper antagonist is a copper binding compound that binds copper¹⁺. In some embodiments, the copper binding compound is a copper chelator. In some embodiments, the copper chelator chelates copper¹⁺. In some embodiments, the copper chelator chelates copper¹⁺ and copper²⁺. In some embodiments, the agent preferentially binds Cu¹⁺. In some embodiments, the agent preferentially binds Cu²⁺. In some embodiments, the agent that preferentially binds Cu²⁺ is triethylenetetramine disuccinate. In some embodiments, the agent binds both Cu¹⁺ and Cu²⁺. In one embodiment, the agent that preferentially binds both Cu¹⁺ and Cu²⁺ is a penicillamine copper chelator, preferably D-penicillamine.

In some embodiments, the pharmaceutical composition used in methods of the invention comprises a therapeutically effective amount of a triethylenetetramine and a pharmaceutically acceptable carrier, glidant, diluent, or excipient. In some embodiments, the triethylenetetramine is in the form of a pharmaceutically acceptable salt.

In one embodiment, the copper-depriving agent or copper antagonist is an agent effective to lower the copper values content or total copper in a subject, and hippocampal copper. In another embodiment, the agent administered to a subject with diabetes and/or vascular dementia comprises or consists essentially of or consists of an agent that binds or chelates copper(II). In another embodiment, the agent comprises or consists essentially of or consists of an agent that binds or chelates copper(I). In another embodiment, the agent comprises or consists essentially of or consists of an agent that binds or chelates both copper(I) and copper(II).

In one embodiment, the copper antagonist or agent effective to lower the copper values content in a subject or otherwise remove excess hippocampal copper comprises or consists essentially of or consists of an agent selected from the group consisting of D-penicillamine; N-acetylpenicillamine; triethylenetetramine (also called TETA, TECZA, trien, triene and trientine), and pharmaceutically acceptable salts thereof; trithiomolybdate, tetrathiomolybdate, ammonium tetrathiomolybdate, choline tetrathiomolybdate; bis-choline tetrathiomolybdate (thiomolybdate USAN, trade name Decuprate), 2,2,2 tetramine tetrahydrochloride; 2,3,2 tetramine tetrahydrochloride; ethylenediaminetetraacetic acid salts (EDTA, a non-preferred non-specific metal binder, administered with care to avoid toxicity); diethylenetriaminetetraacetic acid (DPTA, a non-preferred non-specific metal binder, administered with care to avoid toxicity that is due to chelation of essential metals, such as Zn and Mn); 5,7,7′12,14,14′hexaxmethyl-1,4,8,11 tetraazacyclotretradecane; 1,4,8,11 tetraazacyclotretradecane, including cyclam S, cylams, and copper-chelating cyclam derivatives, e.g., Bn-cyclam-EtOH, oxo-cyclam-EtOH and oxo-Bn-cyclam-EtOH, —(HOCH₂CH₂CH₂)₂(PhCH₂)₂Cyclam and (HOCH₂CH₂CH₂)₂(4-CF₃PhCH₂)₂Cyclam; 1,4,8,11-tetraazacyclotetradecane-1,4,8,11-tetraacetic acid; 1,4,8,11-tetraazabicyclo[6.6.2]hexadecane; 4,11-bis(N,N-diethyl-amidomethyl)-1,4,8,11-tetraazabicyclo[6.6.2]hexadecane; 4,11-bis(amidoethyl)-1,4,8,11-tetraazabicyclo[6.6.2]hexadecane; melatonin; cyclic 3-hydroxymelatonin (3OHM); N(1)-acetyl-N(2)-formyl-5-methoxykynuramine (AFMK); N(1)-acetyl-5-methoxykynuramine (AMK); N,N′-diethyldithiocarbamate; bathocuproinedisulfonic acid; bathocuprinedisulfonate; trimetazidine; triethylene tetramine tetrahydrochloride; 2,3,2-tetraamine; 1,10-orthophenanthroline; 3,4-dihydroxybenzoic acid; 2,2′-bicinchinonic acid; diamsar; 3,4′,5, trihydroxystilbene (resveratrol); mercaptodextran; disulfiram (Antabuse); sarcophagine; DiAmSar; diethylene triamine pentaacetic acid; and calcium trisodium diethylenetriaminepentaacetate; neocuproine; bathocuproine; and carnosine.

In one embodiment, the agent reduces hippocampal copper in the subject. In some embodiments, the agent reduces total copper in the subject. In some embodiments, the copper antagonist or copper-lowering agent maintains total copper in the subject within the normal human serum or plasma range of about 0.8-1.2 milligrams/L, or about 10-25 micromoles/L. In some embodiments, the copper-lowering agent maintains total copper in the subject within at least about 70% of the normal range of about 0.8-1.2 milligrams/L or about 10-25 micromoles/L, e.g., at least about 75%. In some embodiments, the copper-lowering agent maintains total copper in the subject within about 75% to about 85%, or about 85% to about 95% the normal range of copper in human plasma or serum. In some embodiments, one aspect of, the copper status of a subject provided a copper antagonist or other copper-lowering agent or agent to address elevated hippocampal or brain copper in accordance with a method of the invention is determined by evaluating the level or amount of copper in the urine of the subject.

In some embodiments, the compound administered to a subject in carrying out any method of the invention is a triethylenetetramine. In some embodiments, the triethylenetetramine is a hydrochloride salt of triethylenetetramine. In one embodiment, the triethylenetetramine hydrochloride salt is triethylenetetramine dihydrochloride. In another embodiment, the triethylenetetramine hydrochloride salt is triethylenetetramine tetrahydrochloride. In another embodiment, the triethylenetetramine is a succinate salt of triethylenetetramine. In one embodiment, the triethylenetetramine succinate salt is triethylenetetramine disuccinate. In one aspect of the invention, the method employs a crystalline form of triethylenetetramine disuccinate or a hydrochloride salt of triethylenetetramine. In another aspect of the invention, the method employs triethylenetetramine disuccinate anhydrate or a hydrochloride salt of triethylenetetramine anhydrate.

In one aspect of the invention, the method employs a pharmaceutical composition comprising substantially pure triethylenetetramine disuccinate. In another aspect the method employs a pharmaceutical composition comprising substantially pure triethylenetetramine disuccinate and a pharmaceutically acceptable excipient. In another aspect, the method employs a pharmaceutical composition comprising substantially pure triethylenetetramine dihydrochloride or tetrahydrochloride and a pharmaceutically acceptable excipient. A preferred pharmaceutical composition for use in the methods of the invention comprises or consists essentially of or consists of substantially pure triethylenetetramine disuccinate. Another preferred composition comprises or consists essentially of or consists of substantially pure triethylenetetramine disuccinate anhydrate. Another preferred composition is a composition that comprises or consists essentially of or consists of substantially pure triethylenetetramine disuccinate crystal having alternating layers of triethylenetetramine molecules and succinate molecules.

In certain embodiments, the triethylenetetramine succinate salt is a triethylenetetramine disuccinate polymorph. Triethylenetetramine disuccinate polymorphs are described in the art. In certain embodiments, the triethylenetetramine hydrochloride salt is a triethylenetetramine hydrochloride polymorph.

In some embodiments, the invention comprises a method for treating cognitive impairment in a subject with diabetes and excess hippocampal copper, the method comprising administering to said subject a therapeutically effective amount of compound selected from the group consisting of a trientine, a succinic acid addition salt of triethylenetetramine, a hydrochloric acid addition salt of triethylenetetramine, and pharmaceutically acceptable salts of D-penicillamine, N-acetylpenicillamine, tetrathiomolybdate, ammonium tetrathiomolybdate, and choline tetrathiomolybdate. In some embodiments, impaired cognitive function, cognitive decline, and/or dementia or signs of cerebral neurodegeneration is/are treated and/or prevented with these compounds in these subjects, including subjects with type 1 diabetes, type 2 diabetes, type 3 diabetes, type 4 diabetes and/or vascular dementia.

In some embodiments of the methods of the invention, the subject shows signs of cerebral degeneration prior to treatment. In other embodiments, the subject has or is at risk for having impaired cognitive function, cognitive decline, and/or dementia or signs of cerebral neurodegeneration.

In some embodiments, the subject with diabetes and/or vascular dementia has diminished spatial memory or ability to remember directions, locations, and orientations.

In some embodiments of methods of the invention, the method further comprises administering an additional therapeutic agent or agents selected from an anti-inflammatory agent, an agent for treating cardiovascular disease, an agent for treating hypertension, an agent for treating kidney disease, an agent for treating depression, and an agent for treating type 2 diabetes and/or dementia.

In some embodiments, the additional therapeutic agent or agents for treating type 2 diabetes is/are selected from the group consisting of alpha-glucosidase inhibitors, biguanides, dopamine agonists, dipeptidyl peptidase-4 (DPP-4) inhibitors, glucagon-like peptide-1 receptor agonists, meglitinides, sodium-glucose transporter (SGLT) 2 inhibitors, sulfonylureas and thiazolidinediones.

In some embodiments, the additional therapeutic agent or agents for treating dementia is/are selected from the group consisting of cholinesterase inhibitors, antibodies that target the amyloid β protein (a biomarker of Alzheimer disease and other dementias) and N-methyl-D-aspartate (NMDA) receptor antagonists. In some embodiments, the cholinesterase inhibitor is donepezil (Aricept), galantamine (Razadyne, Razadyne ER, Reminyl) or Rivastigmine (Exelon). In some embodiments, the antibody that targets amyloid β protein is Aducanumab-avwa (Aduhelm). In some embodiments, the NMDA receptor antagonist is memantine (Axura, Ebixa, Namenda, etc.).

In some embodiments, the subject is a human.

In some embodiments, the pharmaceutical composition is administered orally in the form of a capsule or tablet.

In some embodiments, the compound is triethylenetetramine dihydrochloride and is administered in an amount of about 1200 mg daily. In some embodiments, the 1200 mg of triethylenetetramine dihydrochloride is administered BID in 600 mg divided doses, TID in 400 mg divided doses, or QID in 300 mg divided doses.

In some embodiments, the compound is triethylenetetramine disuccinate and is administered in a dose ranging from about 2400 mg per day to about 3000 mg per day, or more. In some embodiments, the compound is triethylenetetramine disuccinate and is administered in an amount of about 2800 mg per day. Other useful doses of triethylenetetramine disuccinate are given to equal about 1050 mg/day to about 2300 mg/day, about 1400 mg/day to about 3500 mg/day, about 2400 mg/day to about 3200 mg/day, and about 2800 mg/day to about 5600 mg/day. In some embodiments, these daily triethylenetetramine disuccinate amounts are administered in divided doses.

In another aspect of the invention, the methods of the invention maintain copper levels with about 70% to about 100% of normal in the subject, thereby eliciting by a lowering of copper values in a mammalian patient and/or reducing or lowering the level of copper. in some embodiments, the methods of the invention maintain copper levels with about 70% to about 110% of normal in the subject. Urinary copper output values may be up to about 300-35% of normal soon after treatment is initiated and these and these values typically fall to about 200-250% of normal after 4-12 months.

In certain embodiments, triethylenetetramine disuccinate is administered at an initial dose (or loading dose) followed by a maintenance dose, wherein the loading dose is about or at least 1.5 times greater, about or at least 2 times greater, about or at least 2.5 times greater, or about or at least 3 times greater than the maintenance dose. The maintenance dose may be, for example, about 350 mg, 400 mg, about 500 mg, about 584 mg, about 600 mg and/or about 700 or 701 mg, from 1-4 times per day. In one embodiment, the loading dose is administered once, twice, three, four, or five times before the first maintenance dose, and may be given once, twice, three times or four times a day.

Thus, by way of example, in one embodiment, for a 2337 mg per day triethylenetetramine disuccinate loading dose regimen, triethylenetetramine disuccinate is administered at a daily loading dose (which can be provided in one or several dosages throughout the day) of at least about 3505 mg (1.5×), at least about 4674 mg (2×), at least about 5842 mg (2.5×), or at least about 7001 mg (3×). In one embodiment, the triethylenetetramine disuccinate loading dose is administered in two doses a day, and optionally over 1, 2, 3, 4 or 5 or more days. Other triethylenetetramine disuccinate loading doses are calculated accordingly, based on triethylenetetramine disuccinate maintenance doses given daily or in other frequencies, such as, for example, 2804 or other maintenance doses given daily.

In one embodiment, the triethylenetetramine disuccinate or other copper antagonist described herein is administered twice per day (BID) to provide the desired per day dosing. In another embodiment, the triethylenetetramine disuccinate or other copper antagonist described herein is administered three times per day (TID) to provide desired per day dosing. In a still further embodiment, the doses are administered four times per day (QID) to provide desired per day dosing.

The triethylenetetramine disuccinate doses described and claimed selectively bind to or modify copper(II) values and are used to prevent or reduce or normalize blood and/or tissue copper levels and to prevent and/or reduce unwanted copper accumulation in the hippocampus, and are administered to a subject with elevated hippocampal copper (or at risk elevated hippocampal copper and impaired cognitive function, cognitive decline, and/or dementia or signs of cerebral neurodegeneration.

Triethylenetetramine disuccinate include prodrugs thereof, with doses modified to account for the molecular weight of the “pro-” portion of the triethylenetetramine disuccinate prodrug.

The doses of triethylenetetramine disuccinate or another copper antagonist may be administered alone or in combination with one or more additional ingredients and may be formulated into pharmaceutical compositions including one or more pharmaceutically acceptable excipients, diluents and/or carriers. In some embodiments, the invention provides a combination product comprising (a) a dose of triethylenetetramine disuccinate or another copper antagonist, and (b) a therapeutic agent comprising one or more anti-dementia agents and/or diabetic agents, wherein the components (a) and (b) are adapted for administration simultaneously or sequentially. In some embodiments, component (b) is a an anti-inflammatory agent, an agent for treating cardiovascular disease, an agent for treating hypertension, an agent for treating kidney disease, an agent for treating depression, and/or an agent for treating type 2 diabetes. In a particular embodiment of the invention, a combination product in accordance with the invention is used in a manner such that at least one of the components is administered while the other component is still having an effect on the subject being treated. The dose of triethylenetetramine disuccinate or other copper antagonist and the component (b) therapeutic agent may be contained in the same or one or more different containers and administered separately, or mixed together, in any combination, and administered concurrently. Preferably, both or all three of a triethylenetetramine disuccinate (or other copper antagonist) and an anti-diabetic agent and/or other therapeutic agent are combined in a capsule for oral administration.

In another embodiment, a compounds capable or chelating copper or normalizing copper by reducing or increasing copper levels, preferably levels or amounts of copper²⁺, e.g., triethylenetetramine disuccinate, is used in combination with an agent for the treatment of diabetes. Agents for treating diabetes in accordance with the inventions described and claimed herein include alpha-glucosidase inhibitors, biguanides, dopamine agonists, dipeptidyl peptidase-4 (DPP-4) inhibitors, glucagon-like peptide-1 receptor agonists, meglitinides, sodium-glucose transporter (SGLT) 2 inhibitors, sulfonylureas and thiazolidinediones. Alpha-glucosidase inhibitors for use in the inventions described and claimed herein include, for example, acarbose (Precose) and miglitol (Glyset). The most common biguanide is metformin (Glucophage, Metformin Hydrochloride ER, Glumetza, Riomet, Fortamet). Metformin for use in the inventions described and claimed herein can also be combined with other drugs for type 2 diabetes, and is an ingredient in the following medications: metformin-alogliptin (Kazano); metformin-canagliflozin (Invokamet); metformin-dapagliflozin (Xigduo XR); metformin-empagliflozin (Synjardy); metformin-glipizide; metformin-glyburide (Glucovance); metformin-linagliptin (Jentadueto); metformin-pioglitazone (Actoplus); metformin-repaglinide (PrandiMet); metformin-rosiglitazone (Avandamet); metformin-saxagliptin (Kombiglyze XR); and, metformin-sitagliptin (Janumet). Bromocriptine (Cycloset) is a dopamine agonist that can be used in the inventions described and claimed herein. Dipeptidyl peptidase-4 (DPP-4) inhibitors for use in the inventions described and claimed herein include: alogliptin (Nesina); alogliptin-metformin (Kazano); alogliptin-pioglitazone (Oseni); linagliptin (Tradjenta); linagliptin-empagliflozin (Glyxambi); linagliptin-metformin (Jentadueto); saxagliptin (Onglyza); saxagliptin-metformin (Kombiglyze XR); sitagliptin (Januvia); sitagliptin-metformin (Janumet and Janumet XR); and, sitagliptin and simvastatin (Juvisync). Glucagon-like peptide-1 receptor agonists (GLP-1 receptor agonists) for use in the inventions described and claimed herein include: albiglutide (Tanzeum); dulaglutide (Trulicity); exenatide (Byetta); exenatide extended-release (Bydureon); liraglutide (Victoza); and, semaglutide (Ozempic). Meglitinides for use in the inventions described and claimed herein include: nateglinide (Starlix); repaglinide (Prandin); and, repaglinide-metformin (Prandimet). Sodium-glucose transporter (SGLT) 2 inhibitors for use in the inventions described and claimed herein include: dapagliflozin (Farxiga); dapagliflozin-metformin (Xigduo XR); canagliflozin (Invokana); canagliflozin-metformin (Invokamet); empagliflozin (Jardiance); empagliflozin-linagliptin (Glyxambi); empagliflozin-metformin (Synjardy); and, ertugliflozin (Steglatro). Sulfonylureas for use in the inventions described and claimed herein include: glimepiride (Amaryl); glimepiride-pioglitazone (Duetact); glimepiride-rosiglitazone (Avandaryl); gliclazide; glipizide (Glucotrol); glipizide-metformin (Metaglip); glyburide (DiaBeta, Glynase, Micronase); glyburide-metformin (Glucovance); chlorpropamide (Diabinese); tolazamide (Tolinase); and, tolbutamide (Orinase, Tol-Tab). Thiazolidinediones for use in the inventions described and claimed herein include: rosiglitazone (Avandia); rosiglitazone-glimepiride (Avandaryl); rosiglitazone-metformin (Amaryl M); pioglitazone (Actos); pioglitazone-alogliptin (Oseni); pioglitazone-glimepiride (Duetact); and, pioglitazone-metformin (Actoplus Met, Actoplus Met XR)

Such combination products may be manufactured in accordance with the methods and principles provided herein and those known in the art. Also provided is combination product used in a method as herein described.

For separate or common administration, the copper antagonist, copper chelator or other copper lowering agent formulation, for example, a triethylenetetramine disuccinate formulation, may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. For instance, a stomach-retentive or a mucoadhesive formulation of triethylenetetramine disuccinate can enhance or to extend the absorption of this therapeutic article in the GI tract. A delayed release form of the triethylenetetramine disuccinate will serve to avoid metabolism, prolong and increase absorption, and increase bioavailability by releasing the drug after it passes the stomach. Various different means are available to accomplish these modified release formulations. Such technologies are well-known to one of skill in the art, with specific techniques and excipients selected to address issues or challenges posed by the ADME profile of the article in question.

Mucoadhesive formulations contains specific polymers that adhere to the epithelial lining at the site where they are hydrated. Thus, a drug that is released, for example, in the duodenum after transit through the stomach will adhere to the walls of the GI tract, causing extended and preferential drug release and absorption from this site. Buccal, corneal, respiratory, and vaginal tissues are also lined with mucosal tissues and are thus targets for such formulations. The muco-adhesive properties of most polymers increase with molecular weight, thus MWs in the range of 200,000-700,000, for example, are found to correlate with enhanced muco-adhesion for polyoxyethylene polymers and copolymer. Viscosity, pore size, and the degree of cross linking are other factors that are considered in the selection of muco-adhesive polymers. Hydrogen bonding, flexibility, degree of hydration, and swell are also important factors in drug delivery from muco-adhesive polymers. In addition to polyoxyethylene/polyvinyl alcohol, materials composed of polymeric acrylic and methacrylic esters, and hydroxylated methacrylic polymers are useful for this purpose. Chitosan, cyanoacrylates, hyaluronic acid, hydroxypropyl celluloses, gellan, polycarbopol, and sodium carboxymethylcelluloses are other related polymers have been used in muco-adhesive formulations. Nasal muco-adhesive formulations are developed with attention to the specific properties of such tissues. Nasal delivery system include copolymers of methyl vinyl ether, (hydroxypropyl)methylcellulose (HPMC), sodium carboxymethylcellulose, carbopol-934P and Eudragit RL-10. Mucin, gelatin, polycarbophil, and poloxamer are examples of polymers used for vaginal or rectal muco-adhesive formulations. Oral delivery systems for GI muco-adhesive systems are represented by chitosan, polyacrylic acid, alginate, polymethacrylic acid and sodium carboxymethyl cellulose. Muco-adhesive fixed dose triethylenetetramine disuccinate formulations can be prepared using such compounds.

Stomach retentive formulations are generally designed for drugs that have an optimal window of absorption in the stomach and proximal intestine. Hydrodynamically balanced systems, floating microspheres, gas-generating tablets, formulations that swell to prevent passage from the stomach, and formulations that adhere to the walls of the stomach are examples of such formulations. “Plug” systems that expand to a size that they cannot readily pass the pyloric sphincter are one example of stomach retentive formulations. Low-density (floating) or gas-generating (carbon dioxide) formulations are retained for extended periods of time; such techniques may be used in combination to optimize such performance. Muco-adhesive polymers are also often used to design such an effect into a formulation. Sodium alginate in combination with sodium carbonate or sodium bicarbonate can result in a “rafting” effect such that formulations are retained in the stomach based on buoyancy in the stomach liquid. Stomach-retentive fixed dose triethylenetetramine disuccinate formulations can be prepared using these methods and compounds.

In some embodiments, the copper antagonist or copper-lowering agent is substantially pure, including at least about 90% pure, at least about 95% pure and 100% pure. In some embodiments, the copper antagonist or copper-lowering agent is triethylenetetramine. In some embodiments, the triethylenetetramine is triethylenetetramine disuccinate. In some embodiments, the triethylenetetramine disuccinate is triethylenetetramine disuccinate anhydrate. In some embodiments, the triethylenetetramine disuccinate is crystalline form of triethylenetetramine disuccinate or triethylenetetramine disuccinate anhydrate. In some embodiments of the invention the copper antagonist or copper-lowering agent is a polymorph of triethylenetetramine disuccinate. Triethylenetetramine disuccinate polymorphs are described in, for example, U.S. Pat. No. 8,067,641. In one aspect, the copper antagonist or copper-lowering agent comprises a polymorph of a triethylenetetramine disuccinate wherein the polymorph is a crystal having the structure defined by the co-ordinates of Table 3B found in U.S. Pat. No. 8,067,641. In another aspect of the invention, the copper antagonist or copper-lowering agent comprises a polymorph of triethylenetetramine disuccinate wherein the polymorph is a crystal having the structure defined by the co-ordinates of Table 3C found in U.S. Pat. No. 8,067,641. In other aspects of the invention, the triethylenetetramine disuccinate consists essentially of a triethylenetetramine disuccinate polymorph having a crystal having the structure defined by the co-ordinates of Table 3B in U.S. Pat. No. 8,067,641, or consists essentially of a crystalline triethylenetetramine disuccinate polymorph having the structure defined by the co-ordinates of Table 3C in U.S. Pat. No. 8,067,641.

### Fixed Dose Amounts and Daily or Other Periodic Dosing

Effective fixed triethylenetetramine disuccinate dose amounts are about 400 mg, about 500 mg, about 600 mg or about 700 mg. A fixed dose of 350 mg is also provided. The fixed dose amounts are used, for example, to administer triethylenetetramine disuccinate doses in the range of from about 2400 mg per day to about 3000 mg per day, or other period of time. In one aspect, the effective amount of triethylenetetramine disuccinate is at least about 95% pure, at least about 99% pure, or 100% pure. In another aspect, the effective amount of triethylenetetramine disuccinate is a crystalline form of triethylenetetramine disuccinate. In yet another aspect of this method, the effective amount of triethylenetetramine disuccinate is a triethylenetetramine disuccinate anhydrate. In still another aspect of the method the effective amount of the triethylenetetramine disuccinate is in the form of a fixed dose tablet or capsule. The total dosage may be given in single or divided dosage units (e.g., BID, TID), and preferably maintain normal urine and/or plasma copper levels in a subject, or levels that do not fall below about 70% to 75% of normal. In one preferred embodiment, the fixed doses are administered BID.

### Manufacture

Triethylenetetramine disuccinate suitable for use in the present invention may be obtained from known manufacturing sources or synthesized using methods known in the art. Manufacturing methods are described in U.S. Pat. No. 9,556,123, for example, which describes the synthesis of triethylenetetramines and useful intermediates in their production. U.S. Pat. No. 8,067,641 describes methods for the synthesis of substantially pure triethylenetetramine disuccinate, substantially pure triethylenetetramine disuccinate anhydrate, and triethylenetetramine disuccinate polymorphs.

### Pharmaceutical Preparations

Also provided are pharmaceutical preparations that include a fixed dose of a copper antagonist (for example, a triethylenetetramine, including triethylenetetramine disuccinate) present in a pharmaceutically acceptable vehicle. The term “pharmaceutically acceptable” has the meaning set forth above and includes those vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal.

In one aspect, the present disclosure provides pharmaceutical preparations wherein the copper antagonist (e.g., triethylenetetramine disuccinate), alone or together with another active ingredient, is prepared by combining it (or them) with one or more pharmaceutically acceptable diluents, carriers, adjuvants, and the like in a manner known to those skilled in the art of pharmaceutical formulation. The dosage form can be prepared by combining it with one or more pharmaceutically acceptable diluents, carriers, adjuvants, and the like in a manner known to those skilled in the art of pharmaceutical formulation.

The choice of excipient will be determined in part by the active ingredient, as well as by the particular method used to administer the composition. Accordingly, there is a wide variety of suitable formulations of the pharmaceutical composition of the present invention.

Dosage forms useful herein include any appropriate dosage form known in the art to be suitable for pharmaceutical formulation of compounds suitable for administration to mammals particularly humans, particularly (although not solely) those suitable for stabilization in solutions, tablets or capsules comprising therapeutic compounds for administration to humans.

Compositions may take the form of any standard known dosage form, including those mentioned above, and including tablets, pills, capsules, semisolids, powders, sustained release formulation, solutions, suspensions, elixirs, aerosols, liquids for injection, transdermal delivery devices (for example, a transdermal patch), or any other appropriate compositions. Persons of ordinary skill in the art to which the invention relates will appreciate the most appropriate dosage form having regard to the nature of the condition to be treated and the active agent to be used without any undue experimentation. Various doses and dose ranges, including triethylenetetramine disuccinate doses and dose ranges, are described herein. It should be appreciated that one or more of the other active agents (e.g., an anti-inflammatory agent, etc., and others as described) may be formulated into a single composition with the copper antagonist dose. In certain embodiments, preferred dosage forms include an injectable solution, a topical formulation in a transdermal patch, and an oral formulation. The dosage forms of the invention include any appropriate dosage form now known or later discovered in the art to be suitable for pharmaceutical formulation of compounds suitable for administration to humans.

One example is oral delivery forms of tablet, capsule, lozenge, or the like, or any liquid form, capable of protecting the compound from degradation prior to eliciting an effect, for example, in the alimentary canal if an oral dosage form. Particular formulations for use in the invention are in a solid form, particularly tablets or capsules for oral administration.

Slow- or modified-release copper antagonist preparations (e.g., of triethylenetetramine disuccinate) in tablets or capsules are preferred. Nasal administration to bypass the blood-brain barrier is one method of administration, as well as oral, parenteral, etc.

In addition to standard diluents, carriers and/or excipients, a composition in accordance with the invention may be formulated with one or more additional constituents, or in such a manner, so as to enhance activity or bioavailability, help protect the integrity or increase the half-life or shelf life thereof, enable slow release upon administration to a subject, or provide other desirable benefits, for example. For example, slow-release vehicles include macromers, poly(ethylene glycol), hyaluronic acid, poly(vinylpyrrolidone), or a hydrogel so as to allow for sustained release of the product from the matrix over time. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates will readily identify further additives that may be desirable for a particular purpose.

The copper antagonist doses of the invention may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped (e.g., encapsulated) compound. Liposomes containing copper chelating agents (alone or together with an antiviral and/or anti-inflammatory agent) may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes may be used to encapsulate the triethylenetetramine disuccinate and are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

In one such instance, the desired salt form may be formulated using a gastro-retentive dose form (GRDF). Such delivery forms are formulated with the intent of prolonging gastric retention time, and thus enhancing absorption. Such strategies may employ for instance: 1) passage-delaying agents; 2) large single-unit dosage forms; 3) bioadhesive drug delivery systems; 4) heavy pellets; and 5) buoyant forms. Polymers such a Carbopol, chitosan, sodium alginate, HPMC, polyacrylic acids, polyethylene glycol and modified forms of these polymers are variously used to achieve gastric retention, as a few examples among others.

In a second instance of modified dosage forms for non-immediate release, the product is formulated to delay the release of the drug until after the dosage form exits the stomach. In a delayed release form, the release profile is similar or equal to that of an immediate release form, but the actual release of the drug is delayed by, e.g., enteric coating so that the active ingredient is not release from the dosage form granulation until after transit through the stomach is complete. Enteric coating as one example of this strategy is accomplished by using for instance, (meth)acrylic polymers which do not dissolve in aqueous medium until the pH is above 5.5, thus achieving a dosage form that transits the stomach without releasing the copper antagonist active ingredient.

Extended-release dosage forms are distinct from delayed release in that the release profile of the drug is extended beyond that of an immediate release product. Mechanisms for extended release include delayed dissolution, diffusion, delivery from an intact dosage form by osmotic pressure, maintaining a hydrologic or hydrodynamic balance, and ion exchange. A traditional means of obtaining extended-release delivery is to formulate in a matrix of a non-ionic cellulosic ether (such as HPMC; cf. U.S. Pat. No. 8,865,778B2) in the presence of a selected amount of non-crosslinked swelling agent such as carboxymethyl starch or sodium starch glycolate. Other approaches to achieving the same result are known. For instance, a drug delivery formulation core that contains an osmotic agent and a water-swellable polymer is readily used as a driving force to deliver a drug in a controlled, extended manner.

Therapeutic formulations for use in the methods and preparation of the compositions of the present invention can be prepared by any methods well known in the art of pharmacy. See, for example, Gilman et al. (eds.) GOODMAN AND GILMAN'S: THE PHARMACOLOGICAL BASES OF THERAPEUTICS (8th ed.) Pergamon Press (1990); and Remington, THE SCIENCE OF PRACTICE AND PHARMACY, 20th Edition. (2001) Mack Publishing Co., Easton, Pa.; Avis et al. (eds.) (1993) PHARMACEUTICAL DOSAGE FORMS: PARENTERAL MEDICATIONS Dekker, N.Y.; Lieberman et al. (eds.) (1990) PHARMACEUTICAL DOSAGE FORMS: TABLETS Dekker, N.Y.; and Lieberman et al. (eds.) (1990) PHARMACEUTICAL DOSAGE FORMS: DISPERSE SYSTEMS Dekker, N.Y. Compositions may also be formulated in accordance with standard techniques as may be found in such standard references as Gennaro A R: Remington: The Science and Practice of Pharmacy, 20.sup.th ed., Lippincott, Williams & Wilkins, 2000, for example.

Particular formulations of the invention are in a form for nasal administration, e.g., nanoemulsion. Other formulations of the invention are in the form of a transdermal patch.

### Articles of Manufacture/Kits

The invention also provides an article of manufacture, or “kit”, containing materials useful for treating or preventing treating or preventing impaired cognitive function, cognitive decline, and/or dementia or signs of cerebral neurodegeneration in a subject with diabetes, for example, type 2 diabetes and/or vascular dementia (e.g., VCI), comprising: (a) a pharmaceutical composition comprising a copper antagonist compound capable of reducing, lowering and/or normalizing hippocampal copper levels or amounts; and (b) instructions for use in the therapeutic treatment or prevention of impaired cognitive function, cognitive decline, and/or dementia or signs of cerebral neurodegeneration in a subject with diabetes and/or vascular dementia (e.g., VCI). In some embodiments, the subject has type 1, type 2, type 3 or type 4 diabetes mellitus. In some embodiments, the subject of the instructions has vascular dementia, with or without diabetes.

The kit comprises a container with a composition comprising or consisting essentially of a dose of a copper-lowering agent, for example, triethylenetetramine disuccinate, preferably substantially pure triethylenetetramine disuccinate anhydrate. The kit may further comprise a label or package insert, on or associated with the container (or noted to be available online or in the cloud, or in a flash drive or another storage mechanism). The term “package insert” is used to refer to instructions customarily included in commercial packages of therapeutic products (or available online), that contain information about the indications, usage, dosing, administration, contraindications and/or warnings concerning the use of such therapeutic products. Suitable containers include, e.g., bottles, blister packs, etc. The container may be formed from a variety of suitable material, including plastic, for example. The container may also be a package containing a composition in the form of a tablet or capsule, the latter being one preferred form, where the copper antagonist or copper-lowering agent (e.g., triethylenetetramine disuccinate) is provided in a blister pack, by way of example. The label or package insert indicates that the composition is used for treating subject having diabetes and/or vascular dementia and having (or suspecting of having) impaired cognitive function, cognitive decline, and/or dementia or signs of cerebral neurodegeneration relating to excess or unwanted levels of hippocampal copper. In one embodiment, the instructions recite that the copper antagonist or copper-lowering agent (e.g., triethylenetetramine disuccinate) is to be administered to patients with diabetes and impaired cognitive function, cognitive decline, and/or dementia or signs of cerebral neurodegeneration who are also receiving another therapeutic agent for cardiovascular disease, hypertension, diabetes, kidney disease, inflammation, depression, and/or dementia, etc. The instructions may refer to one or more of the doses or dosing regimens described herein.

In some embodiments, the compound in the kit is selected from the group consisting of triethylenetetramine dihydrochloride, triethylenetetramine tetrahydrochloride and triethylenetetramine disuccinate. In some embodiments, the triethylenetetramine disuccinate in the kit is triethylenetetramine disuccinate anhydrate, preferably substantially pure triethylenetetramine disuccinate anhydrate.

In one aspect, the invention comprises an article of manufacture comprising a package insert instructing the user to administer the copper antagonist compound capable of reducing or lowering copper levels or amounts in the subject, e.g., reducing copper values, reducing total copper and/or normalizing copper levels or amounts and/or copper metabolism, including one or more of the compounds described herein, including copper(I) and/or copper(II) chelators, to a patient with diabetes and a condition or disorder characterized by dementia (or risk for dementia). The compound will address excess copper in the hippocampus or elsewhere in the brain of a subject. The condition or disorder characterized by dementia in the patient with diabetes may be Alzheimer's disease. The condition or disorder characterized by dementia in the patient with diabetes may be vascular dementia.

In a further aspect, the co-existing disease in a dementia patient treatable with a compound capable of lowering copper and/or normalizing copper metabolism (e.g., one or more of the copper chelators or copper-lowering agents described herein) is characterized by excess or reduced copper, such as type 2 diabetes or Alzheimer's, respectively. Thus, in a further aspect, the co-existing disease in a dementia patient treatable with a compound capable of lowering and/or normalizing copper levels or amounts and/or copper metabolism (e.g., one or more of the copper chelators described herein) is characterized by a copper deficiency, such as Alzheimer's disease. In another aspect, the disease, condition or disorder is selected from the group consisting of diabetes mellitus, Alzheimer's disease, Parkinson's disease and Huntington disease.

In another embodiment of the invention, the article of manufacture comprises a container, a label and a package insert. Suitable containers include, for example, bottles, blister packs, etc. The containers may be formed from a variety of materials such as glass or plastic. The container holds a dose(s) of a copper antagonist or other copper-depriving or removing agent composition (e.g., triethylenetetramine disuccinate) effective for treating impaired cognitive function, cognitive decline, and/or dementia or signs of cerebral neurodegeneration and one or more of the symptoms described herein. The label on, or associated with, the container indicates that the copper antagonist or other copper-depriving or removing agent dose composition (e.g., triethylenetetramine disuccinate) is used for a treatment as described herein. In certain embodiments, the patient has type 2 diabetes. In some embodiments, the patient has another form of diabetes. In some embodiments, the patient has type 1 or type 3 diabetes. In certain embodiments, the patient has heart failure. In certain embodiments, the patient has diabetic cardiomyopathy. In certain embodiments, the patient has left ventricular hypertrophy. The package insert may optionally contain some or all of the clinical trial results found on clinicaltrials.gov, for example, or that are later published.

Evaluation of therapy with a copper antagonist or other copper-decreasing or removing agent (e.g., triethylenetetramine disuccinate) may be accomplished by reference to available copper values in mammals (including human beings). Reference herein to “elevated” in relation to the presence of copper values will include humans having at least about 10 mcg free copper/dL of serum when measured. A measurement of free copper equal to total plasma copper minus ceruloplasmin-bound copper can be made using various procedures. A preferred procedure is disclosed in the Merck & Co datasheet (www.Merck.com) for SYPRINE (trientine hydrochloride) capsules, a compound used for treatment of Wilson's Disease, in which a 24-hour urinary copper analysis in is undertaken to determine free cooper in the serum by calculating the difference between quantitatively determined total copper and ceruloplasmin-copper.

### EXAMPLES

The aim of this study was to evaluate the hippocampal copper in Type 2 diabetes (T2D) and sporadic Alzheimer's disease (sAD). The results from these experiments showed, unexpectedly, that brain hippocampal copper is markedly elevated in type 2 diabetes, approximating literature values in Wilson's disease, whereas, contrastingly, hippocampal copper values in the brains of sporadic Alzheimer's disease patients are severely deficient, supporting the use of therapeutic copper-normalizing approaches for treating patients with diabetes who show or are at risk for impaired cognitive function or decline or show signs of cerebral neurodegeneration or vascular dementia.

### Methods

Ethics. All experiments were performed in accordance with relevant guidelines and regulations as stated below. The studies of post-mortem human brain tissue were approved by the University of Manchester Research Ethics Committee. Informed consent for collection of tissues for the T2D/control study was provided by the National Disease Research Interchange (NDRI, Philadelphia, PA), which hosts the Human Tissues and Organs for Research Resource Program funded by the National Institutes of Health (www.ndriresource.org).

Acquisition and Sampling of Human Brain. Regional tissues (frontal cortex, temporal cortex, hippocampus, and related meninges) from T2D cases and matched controls were obtained from the NDRI and transferred to the University of Auckland where they were dissected by neuroanatomists and stored at −80° C. until processing. Wet-weight aliquots of 50±5 mg were: dissected using a ceramic scalpel to avoid metal contamination; dried to constant weight in a centrifugal concentrator (Savant Speedvac™; Thermo-Fisher, Waltham, MA) and dry weights determined by weighing with an analytical balance (DV215CD; Ohaus, Northamptonshire, UK). Dry-weight measurements, as used here, are preferred for the determination of tissue metal levels in clinical laboratories (e.g. for measurement of hepatic Cu for the diagnosis of WD) and are also commonly employed for brain-metal measurements (Xu J, Church Si, Patassini S, et al. Evidence for widespread, severe brain copper deficiency in Alzheimer's dementia Metallomics 2017; 9: 1106-19) and post-mortem metal levels thus determined are found to be stable, robust and replicable (Scholefield M, Church Si, Xu J, et al. Evidence that levels of nine essential metals in postmortem human-Alzheimer's-brain and ex vivo rat-brain tissues are unaffected by differences in postmortem delay, age, disease staging, and brain bank location. Metallomics 2020; 12: 952-62).

Diagnosis and Severity. T2D cases were diagnosed by clinical history whereas corresponding controls had no ante-mortem evidence of diabetes. Neither cases nor controls had historical or post-mortem evidence of dementia or other brain disease. The presence of cognitive impairment was not recorded in the NDRI meta-data nor was it excluded by formal mental state examination. For the AD/control study, diagnosis and severity of sAD were determined by a consultant neuropathologist as described in Xu J, et al. Evidence for widespread, severe brain copper deficiency in Alzheimer's dementia Metallomics 2017; 9: 1106-19. Group characteristics for both T2D/control and AD/control cohorts are as shown in Table 1 and individual NDRI patient characteristics, including age and post-mortem delay in Table 2.

TABLE 1
NDRI sample group characteristics

| | | |
|---|---|---|
| Variable | Control | T2D |

| | | |
|---|---|---|
| Number | 6 | 6 |

| | | | | |
|---|---|---|---|---|
| Age (y) | 76 | (69-78) | 70 | (66-75) * |
| Post-mortem delay (h) | 11 | (5.6-14.2) | 9 | (4.2-17.4) |
| Male sex, n (%) | 3 | (50) | 3 | (50) |

Values are age and post-mortem delay.
p < 0.05 compared with controls; all other differences are non-significant.

TABLE 2
Individual patient characteristics of T2D cases and matched controls

| NDRI | | Age at | | PMD | |
|---|---|---|---|---|---|
| number | Diagnosis | death | Cause of death | (h) | Sex |
| ND05738 | Non-diabetic | 78 | Complications due to CVA | 6.7 | F |
| ND05745 | Non-diabetic | 76 | Stomach cancer with unknown | 5.8 | M |
| ND05764 | Non-diabetic | 76 | AAA rupture | 14.2 | F |
| ND06063 | Non-diabetic | 79 | Cardiac arrest | 8.7 | M |
| ND06116 | Non-diabetic | 69 | Tonsil cancer | 5.6 | M |
| ND08354 | Non-diabetic | 77 | Respiratory failure | 12.5 | F |
| ND05498 | T2D | 69 | Colonic adenocarcinoma | 8.5 | M |
| ND05499 | T2D | 70 | Myocardial infarction | 17.4 | F |
| ND06157 | T2D | 70 | Endometrial cancer | 11.5 | F |
| ND07412 | T2D | 66 | Pulmonary embolism | 12.9 | M |
| ND07636 | T2D | 70 | ESRD | 4.2 | M |
| ND08151 | T2D | 75 | Myocardial infarction | 9.9 | F |

Abbreviations: AAA: Abdominal aortic aneurysms; CVA: Cerebrovascular accident; ESRD: End-stage renal disease; PMD: Post-mortem delay T2D: Type-2 diabetes.
Causes of death were the primary causes listed on the death certificate.

Tissue Digestion. Prior to digestion, all samples were briefly centrifuged at 2400×g (Heraeus Pico 17 Centrifuge; Thermo Fisher Scientific, MA, US) to ensure that tissue aliquots sat at the bottom of the tubes. Concentrated nitric acid (A509 Trace Metal Grade; Fisher, Loughborough, UK) and 5% Agilent Internal Standard mixture (5183-4681; Agilent Technologies, Cheadle, UK) were combined to make the tissue digestion mixture ((Scholefield M, Church Si, Xu J, et al. Evidence that levels of nine essential metals in postmortem human-Alzheimer's-brain and ex vivo rat-brain tissues are unaffected by differences in postmortem delay, age, disease staging, and brain bank location. Metallomics 2020; 12: 952-62). Calibration standards were prepared to the appropriate dilutions (Table SI) using Environmental calibration standard mixture (Agilent 5189-4688) and 2% (v/v) nitric acid digestion solution. For these dry-weight analyses, 200 I.L1 of digestion solution was added to each sample including two empty 2-ml microcentrifuge tubes as digestion blanks. Tube lids were punctured with a septum remover to prevent pressure build-up before transfer into a Dri-Block DB3 heater (Techne, Staffordshire, UK) at room temperature. Temperature was set to 60° C. for 30 min and then increased to 100° C. for a further 3.5 h. Thereafter, 100 1.1.1 of each sample or blank was added to 5 ml of LC/MS grade water in 15-ml Falcon tubes (Greiner) and samples retained at room temperature pending ICP-MS analysis.

ICP-MS. Metal concentrations were determined using an Agilent 7700×ICP-MS spectrometer equipped with a MicroMist nebulizer (Glass Expansion, Melbourne, Australia), a Scott double-post spray chamber and nickel sample and skimmer cones. Samples were introduced into the spray chamber using an Agilent integrated autosampler (I-AS). Before each analysis, the peristaltic-pump sample tubing was replaced to limit abnormal sample delivery to the nebulizer. ICP-MS system optimization and performance reports were generated on Agilent MassHunter Workstation software (G7201A, A.01.01) prior to each analysis to ensure consistent system performance.

To remove spectral interferences, two collision-cell gas modes were employed. All elements were analyzed in helium mode (5.0 ml min⁻¹ He), except for Se which was analyzed in high-energy helium mode (HEHe; 10 ml min′ He) following Agilent's recommendation to reduce interference by polyatomic ion formation. Germanium and indium internal standards were analyzed in both modes. Integration times for relevant trace metals were 3 s for Se; 0.01 s for Fe; 0.03 s for Mn, Cu, and Zn; and 0.1 s for Na, Mg, K, and Ca. A multi-element method using serial dilutions of environmental calibration standards (Table 6; Agilent 5183-4688) was implemented for each analytical batch. 50 μg/1 and 5 μg/1 internal standard calibration standard solutions were used as periodic quality controls 1 and 2, respectively. The limit of quantification, detection limit, and background equivalent concentrations for each trace-metal analyzed in this report were automatically generated by Agilent MassHunter software.

TABLE 6
ICP-MS calibration standard solutions

| Internal standard | | |
|---|---|---|
| solution | | Solution |
| 100 μg/L internal standard | 10 mL 2% nitric acid solution | 100 μL environmental calibration |
| | | standard mixture |
| 50 μg/L internal standard | 15 mL 2% nitric acid solution | 75 μL environmental calibration |
| | | standard mixture |
| 20 μg/L internal standard | 4 mL 2% nitric acid solution | 1 mL 100 μg/L solution |
| 10 μg/L internal standard | 9 mL 2% nitric acid solution | 1 mL 100 μg/L solution |
| 5 μg/L internal standard | 9 mL 2% nitric acid solution | 1 mL 50 μg/L solution |
| 2 μg/L internal standard | 4 mL 2% nitric acid solution | 1 mL 10 μg/L solution |
| 1 μg/L internal standard | 9 mL 2% nitric acid solution | 1 mL 10 μg/L solution |
| 0.5 μg/L internal standard | 9 mL 2% nitric acid solution | 1 mL 5 μg/L solution |
| Blank | 10 mL 2% nitric acid solution | |

Data Analysis. ICP-MS datasets were first exported to individual Microsoft Excel (2010) worksheets where they were corrected for sample weight and dilution and then converted to units of mmol/kg or μmol/kg as appropriate. Means (±95% CI) were calculated and the significance of between-group differences determined by unpaired Welch's (-tests to allow for unequal variances and sample sizes. Statistical calculations were performed using Prism v8.1.1 (GraphPad; La Jolla, CA). P-values <0.05 have been considered significant. To identify cluster separation between T2D metals and the AD metal dataset (Xu J, Church Si, Patassini S, et al. Evidence for widespread, severe brain copper deficiency in Alzheimer's dementia Metallomics 2017; 9: 1106-19) multivariate principal component analysis (PCA) and partial least squares-discriminant analysis (PLS-DA) were applied using the R-platform MetaboAnalyst (www.metaboanalyst.ca). Chong J, Wishart D S, Xia J. Using MetaboAnalyst 4.0 for Comprehensive and Integrative Metabolomics Data Analysis. Curr Protoc Bioinformatics 2019; 68(1): e86.

The average values of both T2D technical-replicate analyses were calculated for each regional analysis and used thereafter for all subsequent PCA and PLS-DA analysis. To assess the suitability of each dataset for PCA, the Kaiser-Meyer-Olkin measure for sampling accuracy and Bartlett's test of adequacy were first performed using SPSS version 23 (IBM; Armonk, NY). Prior to multivariate analysis, all metal datasets were mean-centered and divided by the standard deviation of each variable. Metals with variable importance in projection (VIP) scores based on the PLS-DA model of >I were considered to contribute to group separation.

### Results

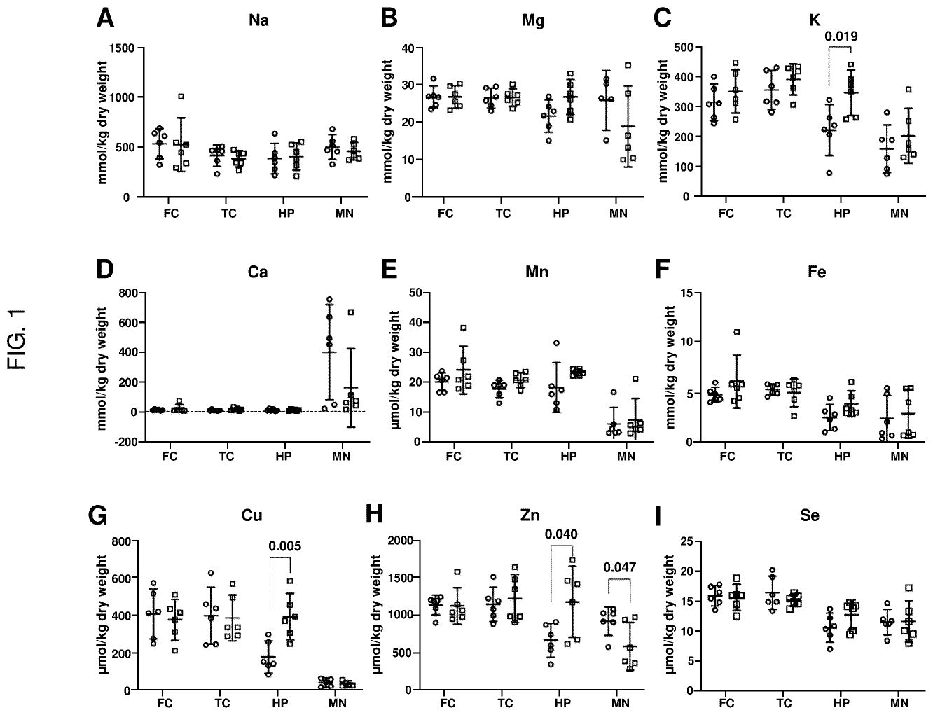

Results showed that Hippocampal copper was substantively increased in the six replicate T2D cases compared with the six matched controls (FIG. 1). This interpretation is further supported by the similar results for copper in the two sequential technical-replicate analyses (P=0.005 and P=0.007, respectively). The results provide robust evidence for the reproducibility of the hippocampal Cu values in this data set (FIG. 1; Tables 3 & 4). See National Academies of Sciences, Engineering, and Medicine. Reproducibility and Replicability in Science: Washington, DC: The National Academies Press; 2019.

TABLE 3
Multi-regional dry-weight metal concentrations for T2D and control brains (run 1)

| Element | | | | | |
|---|---|---|---|---|---|
| (concentration unit) | | Frontal cortex | Temporal cortex | Hippocampus | Meninges |
| Na | Control | 530.1 (379.1-681.1) | 412.7 (305.7-619.6) | 383.6 (231.1-536.1) | 497.3 (374.1-620.5) |
| (mmol/kg) | T2D | 522.8 (254.9-790.6) | 381 (300.1-461.8) | 402.8 (266.6-539.0) | 456.4 (368.2-544.7) |
| Mg | Control | 26.6 (23.6-29.5) | 26.3 (23.6-29) | 21.5 (17.2-25.8) | 25.7 (17.8-33.7) |
| (mmol/kg) | T2D | 26.6 (23.6-29.6) | 26.4 (26.1-28.8) | 26.6 (21.9-31.2) | 18.7 (7.9-29.5) |
| K | Control | 314.7 (254-375.4) | 355.6 (291.3-419.9) | 222.5 (138.1-360.9) | 160.2 (80.7-239.8) |
| (mmol/kg) | T2D | 351 (278.8-423.3) | 390.7 (339.6-441.9) | 346.1 (270.5-421.6)* | 203.3 (112.1-294.6) |
| Ca | Control | 10.17 (6.49-13.85) | 7.93 (4.18-11.69) | 9.70 (3.20-16.21) | 398.43 (76.72-717.1) |
| (mmol/kg) | T2D | 19.36 (7.49-46.22) | 13.87 (5.22-22.52) | 9.75 (4.78-14.72) | 159.34 (−103.5-422.2) |
| Mn | Control | 19.9 (16.9-23) | 17.54 (14.6-20.5) | 18 (9.7-26.3) | 5.7 (0.3-11.5) |
| (μmol/kg) | T2D | 23.93 (15.86-32.01) | 20.47 (17.98-22.95) | 23.1 (22-24.2) | 7.2 (0.03-14.3) |
| Fe | Control | 4.79 (4.04-5.54) | 5.29 (4.73-5.85) | 2.51 (1.19-3.87) | 2.39 (0.06-4.72) |
| (mmol/kg) | T2D | 6.13 (3.46-8.8) | 4.98 (3.58-6.37) | 3.89 (2.6-5.18) | 2.89 (0.45-5.33) |
| Cu | Control | 409.7 (275.8-543.6) | 400.62 (248.8-522.5) | 180.2 (94-266.5) | 43.6 (17.4-69.8) |
| (μmol/kg) | T2D | 378.2 (269.0-487.5) | 388.6 (266-511.3) | 394.8 (270.9-510.7)** | 37.2 (20.6-53.9) |
| Zn | Control | 1128.6 (996.8-1260.3) | 1140.01 (911.7-1368.3) | 660.8 (435-886.5) | 914.5 (723.6-1105) |
| (μmol/kg) | T2D | 1117.0 (873.2-1360.8) | 1215.55 (895.2-1535.9) | 1172.4 (699.2-1645.7)* | 578.8 (261.7-895.9)* |
| Se | Control | 15.8 (14-17.51) | 16.38 (13.64-19.12) | 10.47 (8.05-12.90) | 11.41 (9.31-13.52) |
| (μmol/kg) | T2D | 15.52 (13.32-17.72) | 15.05 (14.02-16.07) | 12.64 (10.20-15.09) | 11.51 (8.07-14.95) |

| Element | | | Vertebrobasilar |
|---|---|---|---|
| (concentration unit) | | Cerebral arteries | arteries^{†} |
| Na | Control | 426.4 (355-497.8) | 433.3 (234-1-632.5) |
| (mmol/kg) | T2D | 403.1 (366.3-439.9) | 428.1 (317.2-539) |
| Mg | Control | 16.9 (15.1-18.8) | 17.3 (12.1-22.6) |
| (mmol/kg) | T2D | 17.8 (15.1-20.5) | 16.6 (13.4-19.9) |
| K | Control | 274.9 (234.2-315.6) | 252.2 (186.1-318.3) |
| (mmol/kg) | T2D | 229.2 (148.7-309.8) | 236.1 (222.5-249.8) |
| Ca | Control | 86.74 (18.47-155) | 82.13 (−13.76-178) |
| (mmol/kg) | T2D | 72.28 (10.35-134.3) | 73.76 (34.21-113.3) |
| Mn | Control | 8.9 (7.1-10.7) | 9.3 (4.4-14.1) |
| (pmol/kg) | T2D | 8.3 (5.5-11.1) | 8.7 (5.9-11.6) |
| Fe | Control | 7.87 (4.41-11.34) | 9.91 (−3.97-23.8) |
| (mmol/kg) | T2D | 5.75 (2.72-8.77) | 4.98 (−0.65-10.6) |
| Cu | Control | 55.5 (41.5-69.5) | 76.4 (−4.9-157.7) |
| (umol/kg) | T2D | 47.7 (31.2-64.2) | 55 (40.4-69.7) |
| Zn | Control | 1128.9 (849.1-1409) | 1249.2 (767.9-1700) |
| (umol/kg) | T2D | 1326.8 (818.6-1835) | 1149.3 (901.3-1397) |
| Se | Control | 11.12 (9.6-12.65) | 11.18 (8.84-13.53) |
| (umol/kg) | T2D | 11.78 (7.9-15.67) | 12.41 (7.64-17.18) |

Data are means (±95% CI);
P values for significance of between-group differences were calculated by Welch's T-tests based on measurements from control (n = 6) and T2D (n = 6) brains.
*P < 0.05,
**P < 0.01.
^{†}Three samples (2 controls and 1 T2D) were unable to be analysed due to insufficient tissue.

TABLE 4
Multi-regional dry-weight metal concentrations for T2D and control brains (run 2)

| Element | | Frontal | | Temporal | | | | | |
|---|---|---|---|---|---|---|---|---|---|
| (concentration unit) | | cortex | | cortex | | Hippocampus | | Meninges | |
| Na | Control | 554.403 | (406.3-702.5) | 385.490 | (270.5-500.5) | 301.198 | (232.5-2369.9) | 606.422 | (459.3-735.5) |
| (mmol/kg) | T2D | 460.704 | (284.6-636.8) | 361.676 | (294.6-428.8) | 339.824 | (184.1-495.5) | 536.046 | (380.6-691.5) |
| Mg | Control | 26.711 | (20.93-32.49) | 22.927 | (21.25-24.60) | 20.227 | (16.7-23.75) | 47.895 | (−4.942-100.7) |
| (mmol/kg) | T2D | 26.111 | (16.14-36.08) | 21.792 | (19.29-24.30) | 22.450 | (17.51-27.39) | 25.149 | (19.03-31.27) |
| K | Control | 322.909 | (242.3-403.6) | 328.364 | (279.5-377.2) | 217.491 | (182.5-252.4) | 239.301 | (121.0-357.6) |
| (mmol/kg) | T2D | 336.474 | (175.1-497.9) | 326.266 | (279.2-373.4) | 309.524 | (203.4-415.7) | 293.734 | (221.0-366.5) |
| Ca | Control | 8.475 | (6.559-10.39) | 9.327 | (5.004-13.65) | 75.17 | (−50.60-200.9) | 445.541 | (−208.4-1100) |
| (mmol/kg) | T2D | 10.093 | (3.969-16.22) | 7.530 | (5.462-9.598) | 7.632 | (4.760-10.50) | 99.957 | (−19.83-219.7) |
| Mn | Control | 20.054 | (15.80-24.31) | 16.867 | (14.51-19.23) | 14.226 | (8.542-19.91) | 11.153 | (2.052-20.25) |
| (μmol/kg) | T2D | 24.828 | (15.17-34.49) | 19.045 | (14.28-23.81) | 19.998 | (16.52-23.48) | 14.095 | (4.640-23.55) |
| Fe | Control | 4.247 | (3.761-4.733) | 4.323 | (3.488-3.336) | 2.040 | (1.273-2.808) | 2.730 | (0.4892-4.972) |
| (mmol/kg) | T2D | 5.539 | (4.208-6.869) | 4.220 | (3.336-5.105) | 3.489 | (2.133-4.846)* | 4.412 | (2.282-6.541) |
| Cu | Control | 390.293 | (124.6-656.0) | 363.303 | (180.6-546.0) | 134.431 | (69.97-198.9) | 202.876 | (−99.56-505.3) |
| (μmol/kg) | T2D | 392.231 | (269.4-515.0) | 335.535 | (269.2-401.9) | 345.556 | (214.0-477.1)** | 221.850 | (36.06-407.6) |
| Zn | Control | 1117.310 | (810.3-1424) | 1026.665 | (789.2-1264) | 662.821 | (422.3-903.3) | 1278.470 | (688.3-1869) |
| (μmol/kg) | T2D | 1198.489 | (570.6-1826) | 1024.758 | (795.9-1254) | 994.338 | (501.1-1488) | 901.150 | (572.4-1230) |
| Se | Control | 14.972 | (11.63-18.32) | 13.770 | (11.34-1620) | 10.171 | (8.094-12.25) | 16.908 | (14.32-19.49) |
| (μmol/kg) | T2D | 14.887 | (9.065-20.71) | 12.653 | (10.95-14.35) | 12.453 | (8.891-16.02) | 17.845 | (9.439-26.25) |

Data are means (±95% CI); P values for significance of between-group differences were calculated by Welch's T-tests based on measurements from control (n = 6) and T2D (n = 6) brains.
*P < 0.05,
**P < 0.01.

Mean hippocampal copper content was on average 2.2-fold elevated in cases compared with controls (394.8 vs. 180.2 μmol per dry-kg of tissue, respectively; Table 6). By contrast, none of the other eight elements differed consistently between cases and controls in the technical-replicate analyses (FIG. 1; Table 3 and Table 4). In the first technical-replicate run, only Zn differed significantly between cases and controls in any of the other regions (frontal cortex, temporal cortex, and meninges) (Table 3). Cases and controls were matched for PMD (post-mortem delay) and sex (Table 3 and Table 4), whereas diabetic cases were slightly younger (70 y, 66-75 [mean; range]) than controls (76 y, 69-78; p=0.011).

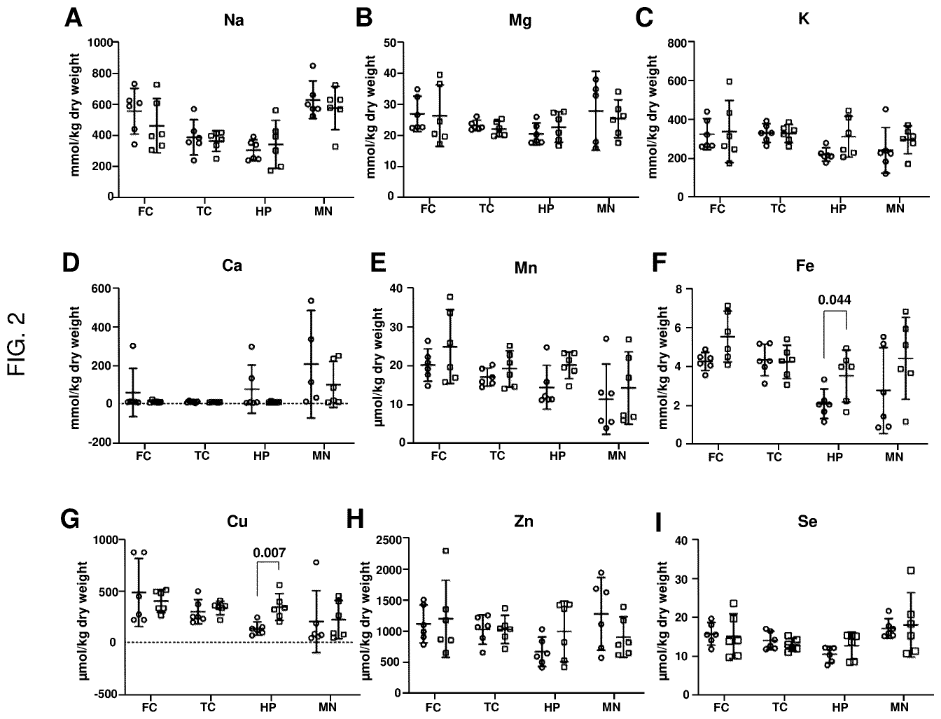

In the second technical-replicate analysis, only mean hippocampal levels of copper (P=0.007) and Fe (P=0.044) and were significantly increased in diabetic cases compared with controls (see FIG. 2). As in the first analysis, the hippocampus displayed the largest concentration difference between case and control copper values. However, for Fe, the largest change in metal levels were in the meninges. No other physiological metals analyzed in this study showed any statistically significant differences between groups.

TABLE 6
Comparison of cerebral copper concentrations and fold-
changes between Wilson's disease, T2D, and controls

| | Dry-weight copper concentrations (μmol/kg) | | | | |
|---|---|---|---|---|---|
| | (also referred to as micromoles/kg) | | | | |
| | | | Wilson's | | Fold- |
| Brain region | Controls | | disease | | change |
| Cortical white matter | 523.8 | (174.6-1301.6) | 2037 | (1730.2-2333.3) | 3.89 |
| Cortical grey matter | 984.1 | (381-1571.4) | 2386.2 | (730.2-4381) | 2.42 |
| Caudate | 1120.6 | (539.7-1492.1) | 2947.1 | (1603.2-5047.6) | 2.63 |
| Thalamus | 936.5 | (492.1-1968.3) | 4470.9 | (3285.7-5063.5) | 4.77 |
| Putamen | 1476.2 | (968.3-1904.8) | 10301.6 | (9603.2-11000) | 6.98 |
| Globus pallidus | 2365.1 | (1666.7-2984.1) | 3772.5 | (1333.3-6333.3) | 1.60 |
| Liver | 1698.4 | (587.3-2730.2) | 13269.8 | (24825.4-8730.2) | 7.81 |

Dry-weight copper concentrations (μmol/kg)

| | Controls | | T2D | | Fold-change |
|---|---|---|---|---|---|
| Hippocampus/ | 180.2 | (86.5-300.8) | 394.8 | (248.9-585.7) | 2.19 |
| Temporal cortex | 400.6 | (244.2-627.4) | 388.6 | (290.8-570.6) | −0.97 |
| Frontal cortex | 409.7 | (251-574.3) | 378.2 | (213.4-512.2) | −0.92 |
| Meninges | 43.6 | (19.7-58.3) | 37.2 | (25.9-58.3) | −0.85 |
| Cerebral artery | 55.5 | (46.9-81.3) | 47.7 | (37.1-66.2) | −0.86 |
| Vertebrobasilar | 76.4 | (40.9-150.7) | 55 | (39-64.9) | −0.72 |
| artery | | | | | |

Data are mean (range) and fold-changes of dry-weight Cu concentrations taken from Cumings (WD)¹⁹ and the present study (T2D). It should be noted that Cu values in Cumings¹⁹ were made using the sodium diethyldithiocarbamate method' (not a reference method). Due to the use of different methodologies employed in these two studies, it was determined that fold-change would be the preferred method by which to compare Cu values between T2D and WD. The method employed for T2D measurements is a current reference method. A measure of significance could not be obtained for the Cumings data as only mean Cu concentrations for controls, and not individual brain concentrations, were provided. Only in the T2D hippocampal region did Cu values differ significantly between cases and controls (P = 0.005 and 0.007 in two sequential technical replicate studies (FIG. 1 and FIG. 2).

Hippocampal copper displayed P-values approximating the three-sigma threshold for both technical replicates, consistent with robust data. See Colquhoun D. An investigation of the false discovery rate and the misinterpretation of p-values. R Soc Open Sci 2014; 1(3): 140216. Furthermore, subsequent statistical-power analysis revealed that copper was the only metal to achieve a power of >0.9 in these studies. Therefore, in the context of statistical validity, this body of evidence places the finding of increased hippocampal copper in T2D at a high level of importance and significance. (see Table 7).

TABLE 7
Power analysis for NDRI dry-weight hippocampal measurements

| | Dry-weight hippocampal | | | Dry-weight hippocampal | | |
|---|---|---|---|---|---|---|
| | tissue (run 1) | | | tissue (run 2) | | |
| | Effect | Actual | Required | Effect | Actual | Required |
| Metal | size | power | sample size | size | power | sample size |
| Na | 0.140 | 0.056 | 1614 | 0.337 | 0.083 | 280 |
| Mg | 1.190 | 0.461 | 26 | 0.543 | 0.137 | 110 |
| K | 1.162 | 0.715 | 16 | 1.222 | 0.481 | 24 |
| Ca | 0.009 | 0.050 | 191084 | 0.581 | 0.149 | 96 |
| Mn | 0.907 | 0.413 | 42 | 1.285 | 0.520 | 22 |
| Fe | 1.111 | 0.808 | 14 | 1.380 | 0.579 | 20 |
| Cu | 2.110 | 0.908 | 10 | 2.14 | 0.915 | 10 |
| Zn | 1.448 | 0.620 | 18 | 0.897 | 0.290 | 42 |
| Se | 0.936 | 0.312 | 38 | 0.821 | 0.251 | 50 |

Test of statistical power for individual NDRI hippocampal dry-weight data. Values highlighted in bold satisfy the statistical power of 80% or the sample size required of ≤ 12. Required samples sizes were generated using a statistical power of 80% and an a error probability of 0.05. Values were determine using G*Power (v. 3.1.9.4) a

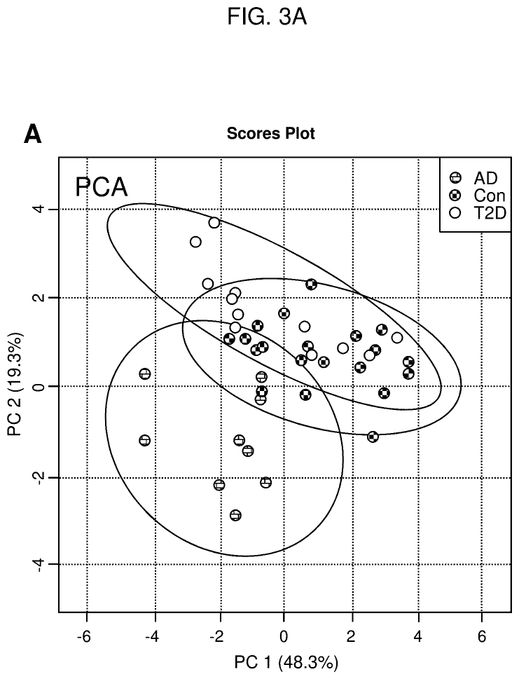

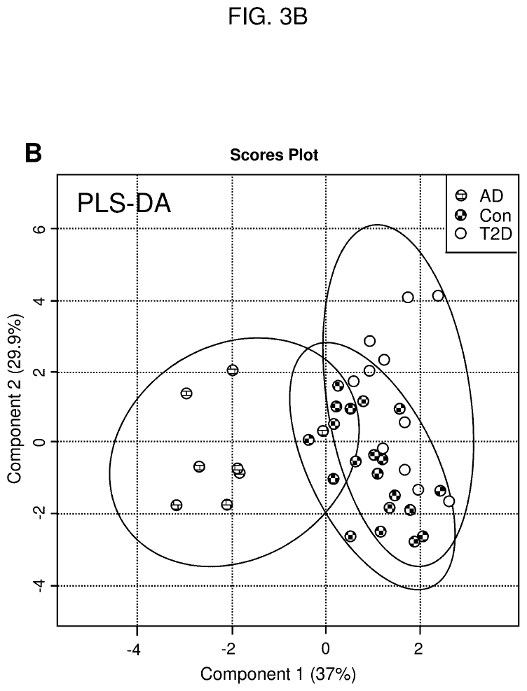

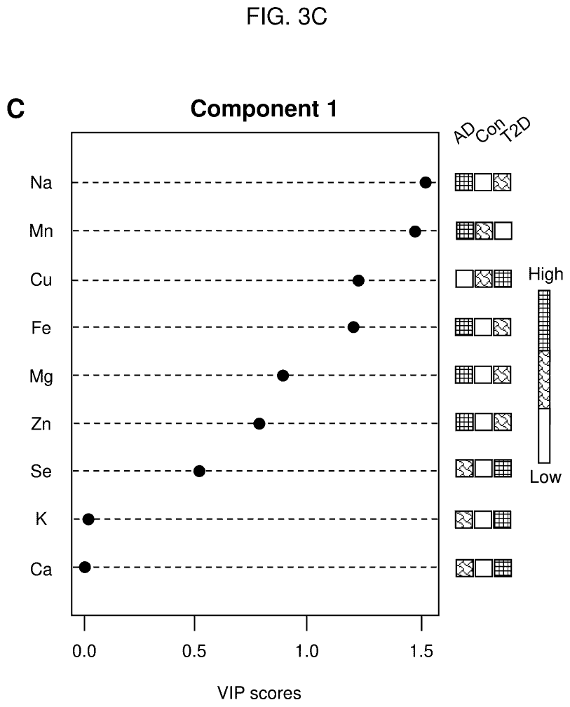

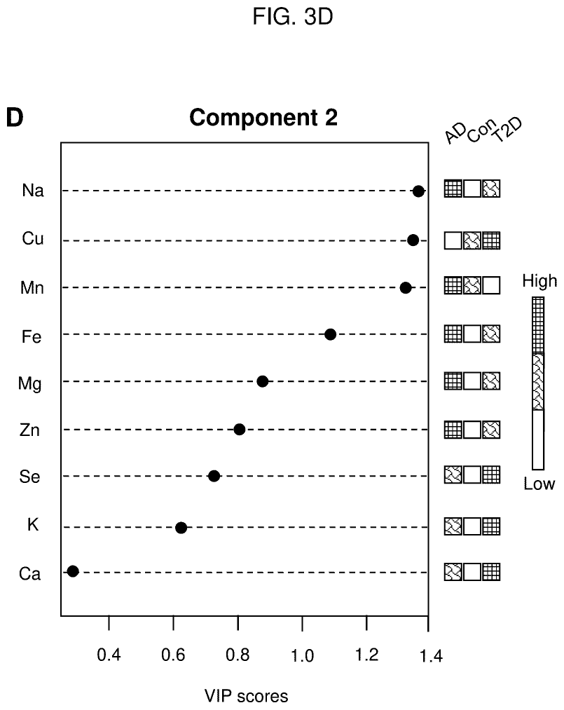

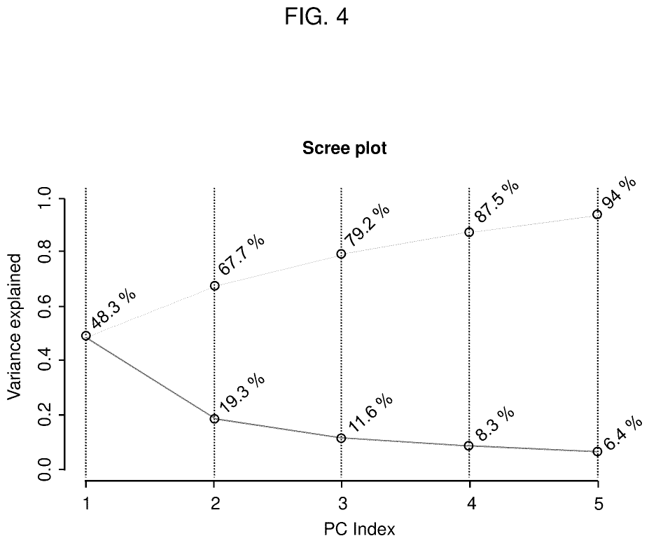

As hippocampal copper was the main essential metal perturbed in the T2D study, multivariate principal component analysis (PCA) was used to further characterize patterns within hippocampal tissue from AD (n=9), T2D (n=6), and control (n=14) brains. Before PCA was performed, the suitability of the analytical methods was assessed. The Kaiser-Meyer-Olkin measure for sampling accuracy for the hippocampal dataset was 0.71, providing robust evidence for the adequacy of these datasets for PCA. Bartlett's test of sphericity was statistically significant (X²(36)=178.313, p<0.001), providing strong evidence for significant correlation between variables to support data reduction. Visual examination of the scree plots revealed that the first two components explained 67.8% of the total variance, thus confirming their utility for two-dimensional PCA (FIG. 4). As PCA plots comparing controls from both the AD and T2D cohorts displayed substantial overlap, both control cohorts were combined and thereafter used as a single control group for comparison against AD and T2D, a process that had been specified a priori. The hippocampal PCA plot revealed nearly complete separation between AD and T2D whereas there was a substantial overlap between T2D and control groups (FIG. 3A), providing further robust evidence for differences in hippocampal metallomic profiles between AD and T2D.

To enhance data discrimination, partial least squares-discriminant analysis (PLS-DA) was additionally performed on the same hippocampal datasets. The PLS-DA model showed a similar pattern to the PCA (FIG. 3B), further supporting the main findings. Based on the PLS-DA model, VIP scores were generated which indicated the relative importance of each metal to the group separation. For principal components 1 and 2; Na, Mn, Cu, and Fe all had VIP scores >1, thus fulfilling the criteria to pinpoint these metals as reliable discriminants within the present hippocampal dataset. Na achieved the highest VIP score for both components, whereas Cu achieved the third and second highest VIP scores in components 1 and 2, respectively (FIG. 3C & FIG. 3D; Table 5).

TABLE 5
Variable importance for projection scores
for the first five principal components

| Metal | PC 1 | PC 2 | PC 3 | PC 4 | PC 5 |
|---|---|---|---|---|---|
| Na | 1.517 | 1.360 | 1.345 | 1.341 | 1.341 |
| Mn | 1.466 | 1.322 | 1.312 | 1.312 | 1.312 |
| Cu | 1.216 | 1.341 | 1.329 | 1.325 | 1.321 |
| Fe | 1.195 | 1.084 | 1.081 | 1.080 | 1.081 |
| Mg | 0.881 | 0.878 | 0.887 | 0.886 | 0.884 |
| Zn | 0.777 | 0.809 | 0.806 | 0.803 | 0.809 |
| Se | 0.515 | 0.724 | 0.716 | 0.717 | 0.719 |
| K | 0.022 | 0.629 | 0.638 | 0.642 | 0.640 |
| Ca | 0.004 | 0.287 | 0.424 | 0.452 | 0.453 |

Values highlighted in bold represent the top two highest scores for each principal component.

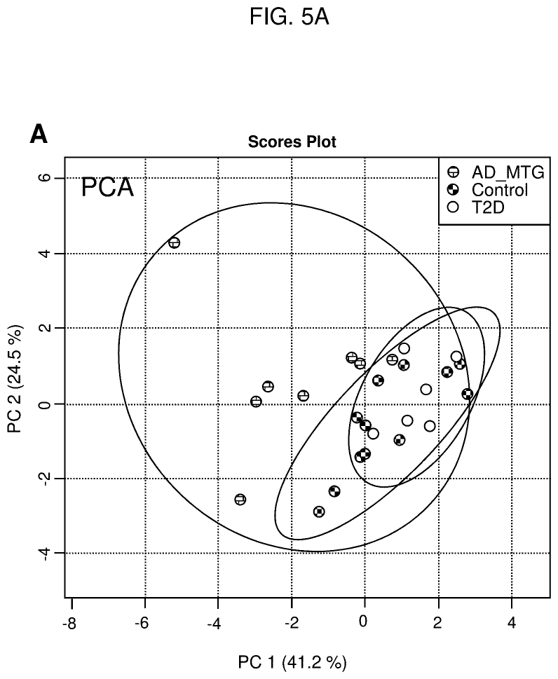

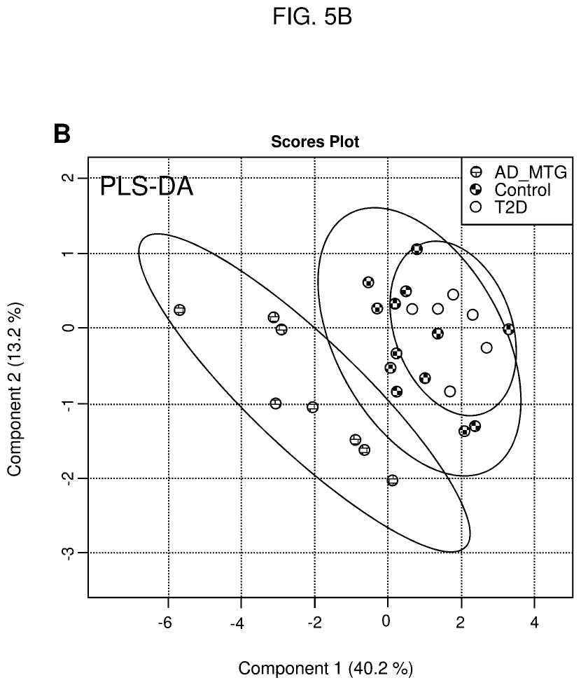

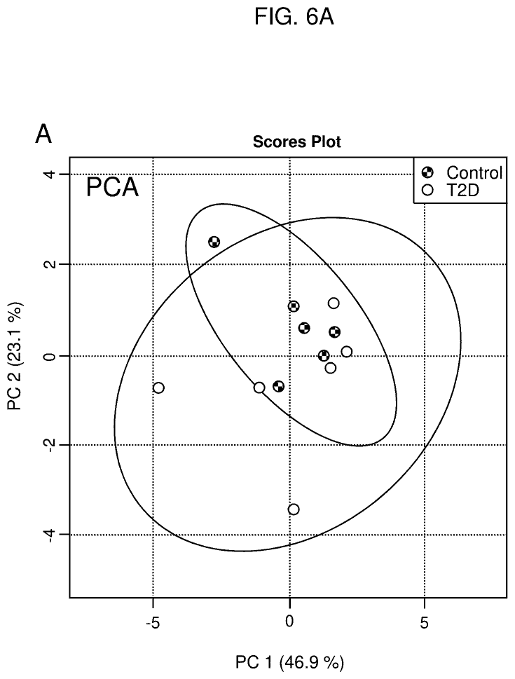

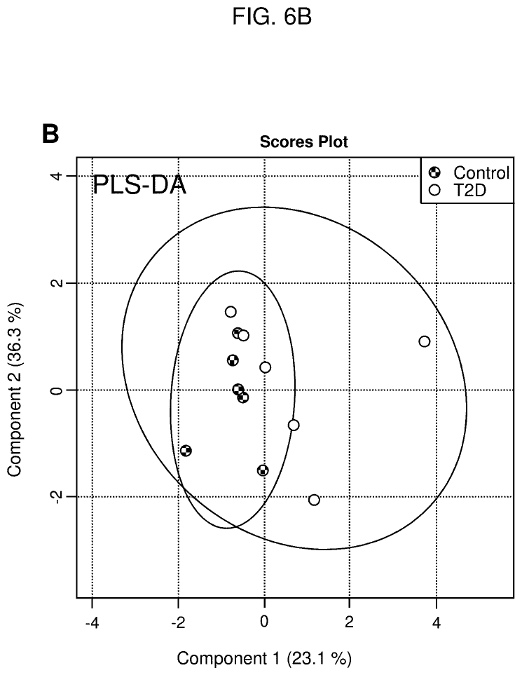

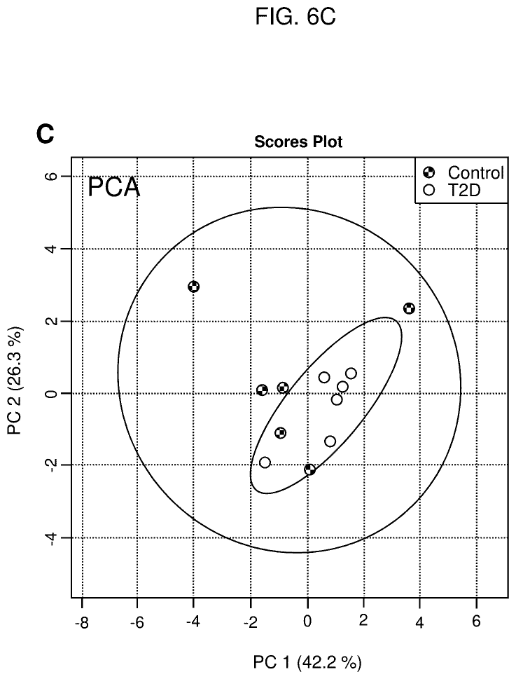

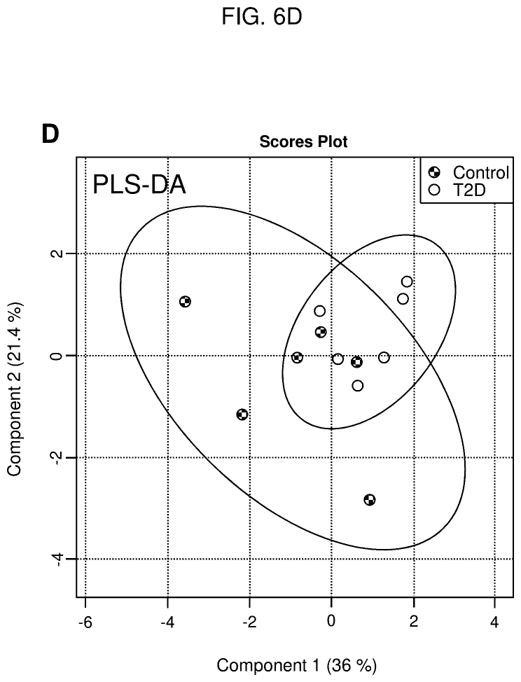

To allow for multi-regional comparisons between PCAs, datasets from frontal and temporal cortices and meninges were also analyzed by applying the same statistical approach as for the hippocampal dataset. Significantly, cluster separation was not detected in any of the remaining three regions (FIG. 5 & FIG. 6). These studies confirm that the disposition of the PCA-derived hippocampal signals differed substantively from those emanating from each of the three other regions (frontal and temporal cortices and meninges). The signal localization in the hippocampus is also consistent with the localization of hippocampal damage in T2D.

### ***

The inventions described and claimed herein have many attributes and embodiments including, but not limited to, those set forth or described or referenced in this Detailed Description. It is not intended to be all-inclusive and the inventions described and claimed herein are not limited to or by the features or embodiments identified in this Detailed Description, which is included for purposes of illustration only and not restriction.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and may not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation agreed to and expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of treating impaired cognitive function in a subject with type 2 diabetes mellitus, comprising administering to the subject a pharmaceutical composition comprising a compound capable of lowering copper in said subject, wherein urinary copper is increased following administration of said composition and impaired cognitive function in said subject is lessened.

2. The method of claim 1, wherein compound is capable of reducing elevated hippocampal copper levels in said subject.

3. The method of claim 1, wherein the pharmaceutical composition comprises a therapeutically effective amount of a triethylenetetramine and a pharmaceutically acceptable carrier, glidant, diluent, or excipient.

4. The method of claim 3, wherein the triethylenetetramine is in the form of a pharmaceutically acceptable salt.

5. The method of claim 4, wherein the triethylenetetramine salt is selected from the group consisting of triethylenetetramine dihydrochloride, triethylenetetramine tetrahydrochloride and triethylenetetramine disuccinate.

6. The method of claim 1, wherein the compound is triethylenetetramine disuccinate.

7. The method of claim 1, wherein the triethylenetetramine disuccinate is a crystalline form of triethylenetetramine disuccinate.

8. The method of claim 1, wherein the triethylenetetramine disuccinate is a triethylenetetramine disuccinate anhydrate.

9. The method of claim 1, wherein the subject shows signs of cerebral degeneration and one or more signs of cerebral degeneration are improved.

10. The method of claim 1, wherein the subject has or is at risk for having cognitive decline and/or dementia and said cognitive decline and/or dementia in said subject is improved.

11. The method of claim 1, wherein the subject has diminished spatial memory or ability to remember directions, locations, and orientations, and the subjects spatial memory and/or ability to remember directions, locations, and/or orientations is improved.

12. The method of claim 1 further comprising administering an additional therapeutic agent or agents selected from an anti-inflammatory agent, an agent for treating cardiovascular disease, an agent for treating hypertension, an agent for treating kidney disease, and an agent for treating type 2 diabetes.

13. The method of claim 12, wherein the agent for treating type 2 diabetes is selected from the group consisting of alpha-glucosidase inhibitors, biguanides, dopamine agonists, dipeptidyl peptidase-4 (DPP-4) inhibitors, glucagon-like peptide-1 receptor agonists, meglitinides, sodium-glucose transporter (SGLT) 2 inhibitors, sulfonylureas and thiazolidinediones.

14. The method of claim 1, wherein the subject is a human.

15. The method of claim 1, wherein the pharmaceutical composition is administered orally in the form of a capsule or tablet.

16. The method of claim 1, wherein the compound is triethylenetetramine dihydrochloride and is administered in an amount of about 1200 mg daily.

17. The method of claim 1, wherein the compound is triethylenetetramine disuccinate and is administered in a daily dose from about 2400 mg per day to about 3000 mg per day of triethylenetetramine disuccinate.

18. The method of claim 1, wherein the compound is triethylenetetramine disuccinate and is administered in an amount of about 2800 mg daily.

19. The method of claim 16, wherein the 1200 mg is administered BID in 600 mg divided doses, TID in 400 mg divided doses, or QID in 300 mg divided doses.

20. The method of claim 18, wherein the 2800 mg is administered in divided doses.

21. A kit for the therapeutic treatment of treating or preventing impaired cognitive function in a subject with type 2 diabetes mellitus comprising: a) a pharmaceutical composition comprising a compound capable of reducing copper levels and/or normalizing copper metabolism in a subject; and b) instructions for use in the therapeutic treatment or prevention of impaired cognitive function in a subject with type 2 diabetes mellitus.

22. A kit according to claim 21, wherein the compound is selected from the group consisting of triethylenetetramine dihydrochloride, triethylenetetramine tetrahydrochloride and triethylenetetramine disuccinate.

23. A kit according to claim 22, wherein the triethylenetetramine disuccinate is triethylenetetramine disuccinate anhydrate.
